(19) United States
(12) Patent Application Publication
(10) Pub. No.: US 2025/0282809 A1
(43) Pub. Date: Sep. 11, 2025

(54) Chemical Synthesis Method for Pseudomonas aeruginosa Serotype O5 O-Antigen Oligosaccharide

(71) Applicant: Jiangnan University, Wuxi (CN)

(72) Inventors: Jian YIN, Wuxi (CN); Jing HU, Wuxi (CN); Guangzong TIAN, Wuxi (CN)

(21) Appl. No.: 19/216,865

(22) Filed: May 23, 2025

Related U.S. Application Data
(63) Continuation of application No. PCT/CN2024/127397, filed on Oct. 25, 2024.

(30) Foreign Application Priority Data
May 10, 2024 (CN) 2024105749848

(51) Int. Cl. C07H 15/12; C07H 1/00

(52) U.S. Cl. CPC C07H 15/12; C07H 1/00

(57) ABSTRACT

Disclosed is a chemical synthesis method for Pseudomonas aeruginosa O5 serotype O-antigen oligosaccharide, which belongs to the field of chemical synthesis. In the disclosure, O-antigen trisaccharide is constructed with a D-glucuronic acid building block and a D-fucosamine building block, where the stereoselective synthesis of a 1,2-α-cis-glycosidic bond of D-fucosamine depends on remote acyl participation and reagent regulation, and synthesis of two types of 1,2-β-trans-glycosidic bonds of 2,3-diaminomannuronic acids is achieved via S_{N}2 nucleophilic substitution of azido at position C2; and via selective assembly of protecting groups, orthogonal modification of modifying groups, and regulation of the reactivity of glycosyl donors and acceptors, multifunctional modified O-antigen target trisaccharide is successfully prepared. According to the method in the disclosure, the raw materials are cheap and easily available, and the preparation method is simple and easy to repeat. Therefore, the method has a very good application prospect in developing vaccines against P. aeruginosa.

### TECHNICAL FIELD

The disclosure relates to a chemical synthesis method for Pseudomonas aeruginosa serotype O5 O-antigen oligosaccharide, which belongs to the field of chemical synthesis.

### BACKGROUND

Pseudomonas aeruginosa, also known as P. aeruginosa, is a type of opportunistic pathogens with strong environmental adaptability and is one of the main pathogens causing hospital infections. P. aeruginosa infection can cause a series of complications such as pyohemia and sepsis, and cause fatal harm to immunocompromised populations such as cystic fibrosis patients, AIDS patients, and post-chemotherapy patients (K. Grimwood et al., Hum Vaccin Immunother 2015, 11, 14-20). According to a report from the Centers for Disease Control and Prevention, approximately 51,000 patients are infected with the P. aeruginosa each year, with a mortality rate of approximately 5.3% (M. E. Chirgwin et al., Materials 2019, 12, 4093). The P. aeruginosa is highly resistant to a variety of antibiotics (C. K. Elogne et al., Afr J Microbiol Res, 2018, 12, 62-67). In the list of “Priority Pathogens” with antibiotic resistance first published by the World Health Organization (WHO) in 2017, the P. aeruginosa was listed as the “Critical Priority” category. The drug resistance problem of the P. aeruginosa has become one of the biggest threats to human health (E. Tacconelli et al., World Health Organization, 2017). Since immunoprophylactic strategies are not affected by the mechanism of antibiotic resistance, there is an urgent need to develop highly effective and safe vaccines for the treatment of drug-resistant pathogens. Researchers have conducted a large number of studies on P. aeruginosa vaccines, mainly including whole-cell vaccines and various subunit vaccines, but no vaccine available to human beings has been approved for marketing (G. P. Priebe et al., Expert Rev Vaccines, 2014, 13, 507-519). Compared to the whole-cell vaccines containing many ineffective antigens, the subunit vaccines such as P. aeruginosa cell surface polysaccharide subunit vaccines and membrane protein subunit vaccines can provide more effective immune protection. Studies have shown that vaccine research is conducted by taking P. aeruginosa lipopolysaccharide as an immunogen, which has shown a relatively good protective effect, and the most effective antigenic target for immunity is an O-antigen polysaccharide moiety of a bacterial surface lipopolysaccharide (LPS) (S. J. Cryz et al., Infect Immun, 1984, 44, 508-513). Therefore, O-antigens of the lipopolysaccharide have been widely used in the development of the P. aeruginosa vaccine (N. F. Abu-Baker et al., Advances in Microbiology, 2016, 6, 332-342).

Based on different O-antigens of the P. aeruginosa lipopolysaccharide, 20 serotypes of P. aeruginosa with different immunological specificities have been identified (P. V. Liu et al., J Clin Microbiol, 1990, 28, 922-925). Among the 20 serotype O-antigens of the P. aeruginosa whose structures have been clearly defined, serotype O5 O-antigens are highly similar to O2, O16, O18 and O20 serotype O-antigens in their trisaccharide repeating fragments. A serotype O5 O-antigen polysaccharide structure is of interest owing to its high structural specificity. The polysaccharide structure consists of a complex modified polyaminotrisaccharide repeating unit: [→4)-β-D-Manp2NAc3AmA-(1→4)-β-D-Manp2NAc3NAcA-(1→3)-α-D-FucpNAc-(1→] (I. Sadovskaya et al., Eur. J. Biochem. 2000, 267, 1640-1650). It is worth noting that polysaccharide with two continuous β-D-ManpN3NA fragments is not common in nature and has not been synthesized before. In addition, most of hydroxyl on saccharide rings of the trisaccharide repeating fragment is substituted with amino, and only one hydroxyl is exposed, thus forming high-density functionalized aminoglycosides. Another special feature is that the trisaccharide repeating fragments are all linked by a 1,2-cis-glucosidic bond. In bacterial O-antigens, there is a unique functional group referred to as acetamidino (Am), which is specifically attached onto the amino at position C3 of 2,3-diamino-D-glucuronic acid of the O-antigen trisaccharide. Although remarkable progress has been made in recent years in stereoselective glycosylation synthesis methods and strategies for assembling complex oligosaccharides, chemical synthesis of high-density functional aminoglycosides is still full of challenges. The difficulties include: it is very difficult to synthesize stereospecific trisaccharides because all linkages are the 1,2-cis-glucosidic bonds; and rare sugar ManpNAc3NAcA has poor reactivity in the process of glycosylation and requires orthogonal assembly of acetamidino and acetyl on the amino. Therefore, for the total synthesis of this structure, an overall route design is required, including key links such as the selection of protecting groups, the selection of the timing of introducing modified groups, and the efficiency and selectivity of the glycosylation reaction.

### SUMMARY

The disclosure relates to synthesis of a P. aeruginosa serotype O5 O-antigen trisaccharide fragment by a chemical method for three sugar building blocks, mainly including a 3-amino-D-glucose building block, a D-glucuronic acid building block, a D-fucosamine building block, along with a 1,2-α-cis-glucosidic bond, a 1,2-β-cis-glucosidic bond, and orthogonal modification of an amino functional group. Construction of the 1,2-cis-glucosidic bond and the orthogonal modification of the amino functional group are key steps for target trisaccharide. In the disclosure, the stereoselectivity problem of constructing the 1,2-α-cis-glucoside bond under the synergistic effect of the remote acyl participation effect and the additive effect is successfully solved. Stereoscopic construction of two types of 1,2-β-cis glycosidic bonds is achieved via S_{N}2 nucleophilic substitution of azido at position C2. Using a synthesized trisaccharide precursor, oxidation of hydroxyl at position C6 and the orthogonal modification of different amino functional groups are carried out, and finally, deprotection is carried out to afford the target trisaccharide, as represented by formula VI. In the disclosure, synthetic oligosaccharides are immobilized to the surface of a microarray through amino linker arms, and oligosaccharide antigens are screened with the serum of a P. aeruginosa patient and applied to the antigenicity research of the synthetic oligosaccharides.

A first objective of the disclosure is to provide a synthesis method for P. aeruginosa serotype O5 O-antigen trisaccharide, where the method includes: constructing O-antigen trisaccharide with 3-amino-D-glucose building blocks, D-glucuronic acid building blocks, and D-fucosamine building blocks; constructing a 1,2-α-cis-D-fucosamine glycosidic bond of D-fucosamine under the synergistic effect of the remote acyl participation effect and the additive effect, and constructing 1,2-β-trans glycosidic bonds of two types of 2,3-di-amino-D-mannuronic acids with an S_{N}2 substitution reaction of azido at position C2; and completing the orthogonal assembly of five amino functionally modified groups in O-antigen trisaccharide.

The disclosure relates to an oligosaccharide fragment in which P. aeruginosa serotype 05 O-antigen trisaccharide is assembled with a linker arm, where the chemical structural formula of this sugar chain can be expressed as general formula (VI):

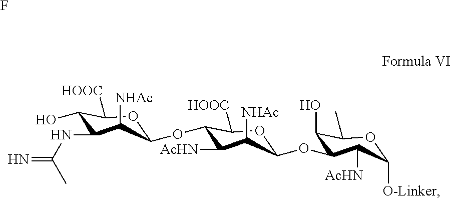

where Linker is -L-NH₂, L representing a linker arm.

In the disclosure, the linker arm may be a chain structure with 2 to 40 carbon atoms that contain 0 to 6 heteroatoms (including carbon atoms on a side chain).

In the disclosure, when the main chain length of the linker arm is 4 to 8 atoms, the chain may include 1, 2, or 3 heteroatoms (0, N, and S). When the main chain length of the linker arm is 9 to 14 atoms, the chain may include 1, 2, 3, 4, 5, or 6 heteroatoms (O, N, and S).

In the disclosure, the linker arm -L- may be a fully or partially fluorine-substituted cyclic structure. The linker arm -L- may include a three-, four-, five- or six-membered saturated carbon ring. The linker arm -L- may also include a five-membered unsaturated carbon ring (nonaromatic ring). The linker arm -L- may also include a four-, five- or six-membered saturated oxygen heterocycle. The linker arm -L- may also include a four-, five- or six-membered saturated nitrogen heterocycle. The linker arm -L- may also include a six-membered aromatic carbon ring.

In the disclosure, the linker arm -L- may also include an amide bond and/or ureido.

In the disclosure, the above-mentioned linker arms -L- may also include one or more substituent groups, which may include: —F, —Cl, —CH₃, —C₂H₅, —C₃H₇, —C₅H₉, —C₆H₁₃, —OCH₃, —OC₂H₅, —CH₂F, —CHF₂, —CF₃, —C(O)—NH₂, —SCH₃, —SC₂H₅, —NHC(O)CH₃, —N(CH₃)₂, and —N(C₂H₅)₂.

In the disclosure, the synthesized sugar chain structure contains basic (ethyl) and acidic (carboxyl) groups which can form corresponding salts with organic or inorganic acids or bases.

In the disclosure, since the synthesized sugar chain structure contains both basic (ethyl) and acidic (carboxyl) groups, intramolecular proton transfer may occur, that is, protons of the acidic groups are transferred to the basic groups, and the general formula may be amphoteric molecules containing —O and —NH₃⁺.

The disclosure provides a chemical synthesis method for a linker arm modified sugar chain (as represented by general formula VI) of P. aeruginosa serotype O5 O-antigen polysaccharide, where three monosaccharide building blocks A, B, and C are taken as raw materials, as represented by formulas (I) to (III), respectively:

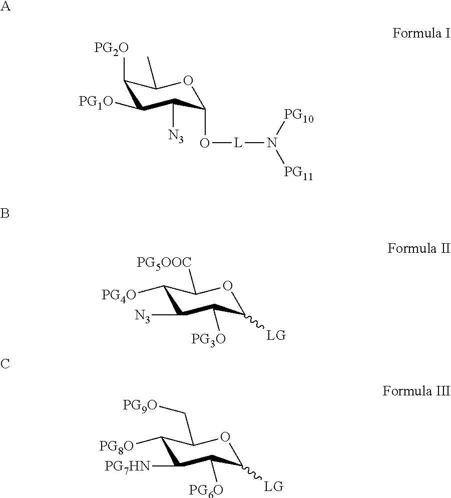

where:

PG₁ is H;

PG2, PG₃, PG₄, and PG₆ are temporary hydroxyl protecting groups, each independently selected from acetyl (Ac), benzoyl (Bz), pivaloyl (Piv), chloracetyl (CIAc), levulinyl (Lev), 9-fluorenylmethoxycarbonyl (Fmoc), allyloxycarbonyl (Alloc), 2-naphthylmethyl (Nap), p-methoxybenzyl (pMB), allyl (All), tert-butyldimethylsilyl (TBS), or triethylsilyl (TES);

PG₅ is a carboxyl protecting group, which may be benzyl (Bn), methyl (Me), ethyl (Et), tert-butyl (tBu), or allyl (All);

PG₇ is a temporary amino protecting group, which may be trichloroethoxycarbonyl (Troc), phthaloyl (Phth), 9-fluorenylmethoxycarbonyl (Fmoc), tert-butyloxycarbonyl (Boc), and the like;

PG₈ and PGg are hydroxyl protecting groups, which may be phenylmethylene (PhCH), naphthylmethylene (NapCH), isopropylidene ketal ((CH₃)₂CH), and the like;

PG₁₀ and PG₁₁ are amino protecting groups, which may be benzyl (Bn) and carbobenzoxy (Cbz); and

LG is a leaving group used for a glycosylation reaction, such as fluorine (F), chlorine (Cl), bromine (Br), iodine (I), trichloroacetimidate (CCl₃C(═NH)O—), N-phenyl trifluoroacetimidate glycoside (CF₃C(═NPh)O—), ethylthio (SEt), phenylthio (SPh), p-tolylthio (STol), dibutylphosphonato (—P(═O)—(OBu)₂), or the like;

the following procedures are included:

(1) carrying out a glycosylation reaction on the monosaccharide building block B (D-glucuronic acid building block) and the monosaccharide building block A (D-fucosamine building block), and carrying out synthesis to obtain a disaccharide fragment represented by formula IV,

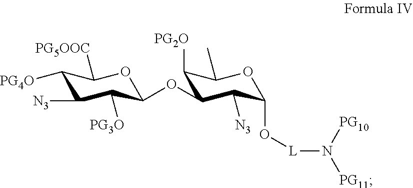

and

(2) deprotecting the resulting disaccharide fragment, removing the protecting group PG₄, and then carrying out glycosylation with the monosaccharide building block C (3-amino-D-glucose building block), and carrying out synthesis to obtain a trisaccharide intermediate fragment represented by formula V,

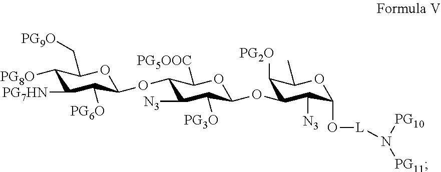

and

(3) reducing the resulting trisaccharide intermediate fragment, deprotecting PG3 and PG₆, and carrying out azidation on deprotected position 2 of the monosaccharide building block B and deprotected position 2 of the monosaccharide building block C; subsequently, reducing positions 4 and 6 of the monosaccharide building block C in the trisaccharide intermediate fragment, removing PG₈ and PG₉, and carrying out oxidization on position 6 to obtain carboxylic acid groups; then, reducing the deprotected position 2 in the monosaccharide building block B and an azide group at position 2 in the monosaccharide building block C to obtain acetylamino; and then, constructing an acetamidine structure with an imine reagent and position 3 in the monosaccharide building block C, and finally, reducing amino in Linker to obtain a target product represented by formula VI. In an embodiment of the disclosure, a synthetic route of the method is as follows:

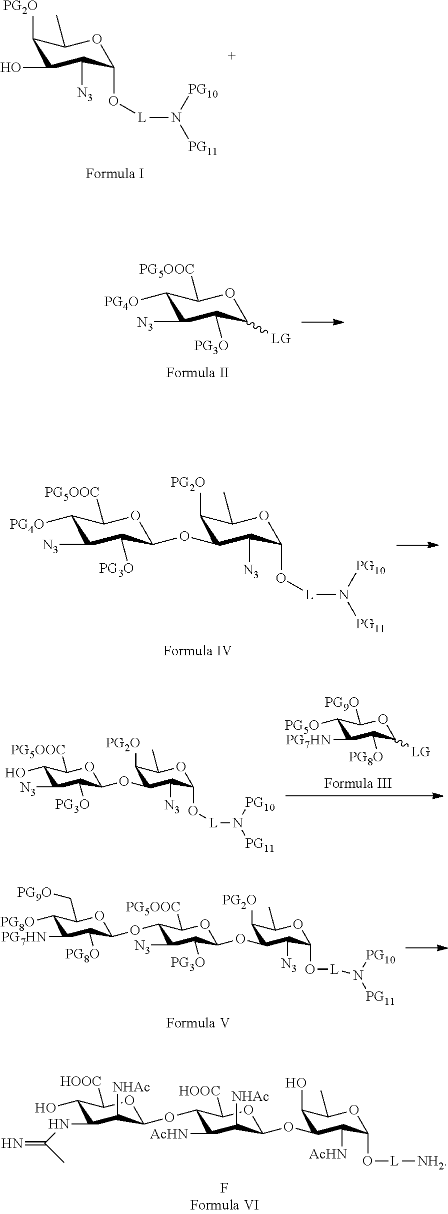

In the disclosure, O-antigen trisaccharide is constructed with a D-glucuronic acid building block and a D-fucosamine building block, where the stereoselective synthesis of a 1,2-α-cis-glycosidic bond of D-fucosamine depends on remote acyl participation and reagent regulation, and synthesis of two types of 1,2-β-trans-glycosidic bonds of 2,3-diaminomannuronic acids is achieved via SN2 nucleophilic substitution of azido at position C2; via selective assembly of protecting groups, orthogonal modification of modifying groups, and regulation of the reactivity of glycosyl donors and acceptors, multifunctional modified O-antigen target trisaccharide and its oligosaccharide mimetics are successfully prepared; and a synthesized O-antigen oligosaccharide fragment is immobilized to the surface of a glass slide to prepare a glycan microarray, and effective antigens are screened with the serum of a patient to determine the epitope of O-antigen oligosaccharide. The raw materials are cheap and easily available, and the preparation method is simple and easy to repeat. Therefore, the disclosure has a good application prospect in developing vaccines against P. aeruginosa.

In an embodiment of the disclosure, the method specifically includes: taking 3,4-O-diacetylfucal as a raw material, under the action of iodobenzene diacetate (Ph (OAc)₂), producing 1-selenophenyl-2-azido fucose by reacting with azidotrimethylsilane (TMS-N₃) and diphenyl diselenide (Ph₂Se₂); removing acetyl under alkaline conditions; selectively protecting hydroxyl at position C3 as 3-ONap, and protecting hydroxyl at position C4 with benzoyl to finally obtain 1,2-α-cis-D-fucosamine glycosidic bond with high stereoselectivity under the synergistic effect of the additive effect (Ph₃PO) and the remote acyl participation effect (Bz).

In an embodiment of the disclosure, the method further includes: taking glucose as a raw material, using trifluoromethylsulfonyl-mediated nucleophilic substitution of azido at position C3 to afford an amino precursor at position C3, and also using S_{N}2 nucleophilic substitution of azido at position C2 to afford an amino precursor at position C2, and completing efficient construction of two types of 1,2-β-trans-glycosidic bonds of 2,3-diaminomannuronic acids.

In an embodiment of the disclosure, the Lewis acid includes borontrifluoride diethyl etherate (BF₃.Et₂O), trifluoromethanesulfonic acid (TfOH), trimethylsilyl trifluoromethanesulfonate (TMSOTf), silver trifluoromethanesulfonate (AgOTf), or the like. The reaction of thioglycoside involves additionally adding NIS and the Lewis acid, both of which serve as accelerators.

In an embodiment of the disclosure, the glycosylation reaction in step (1) is carried out under an atmosphere where an activating reagent Lewis acid, a solvent, and a molecular sieve are present.

In an embodiment of the disclosure, the activating reagent Lewis acid for the glycosylation reaction in step (1) is any one or more of borontrifluoride diethyl etherate (BF₃.Et₂O), trifluoromethanesulfonic acid (TfOH), trimethylsilyl trifluoromethanesulfonate (TMSOTf), silver trifluoromethanesulfonate (AgOTf), or the like.

In an embodiment of the disclosure, the glycosylation reaction in step (2) is carried out under an atmosphere where an activating reagent Lewis acid, N-iodosuccinimide (NIS), a solvent, and a molecular sieve are present.

In an embodiment of the disclosure, a dosage of the activating reagent Lewis acid for the glycosylation reaction in step (2) is 0.1 equivalent (eq) to 1 equivalent (eq), and a dosage of NIS is 1.2 eq to 1.5 eq.

In an embodiment of the disclosure, the activating reagent Lewis acid for the glycosylation reaction in step (2) is any one or more of borontrifluoride diethyl etherate (BF₃.Et₂O), trifluoromethanesulfonic acid (TfOH), trimethylsilyl trifluoromethanesulfonate (TMSOTf), silver trifluoromethanesulfonate (AgOTf), or the like.

An objective of the disclosure is to provide a synthesis method for P. aeruginosa serotype O5 O-antigen trisaccharide assembled with an amino linker arm, and a serotype O5 O-antigen oligosaccharide fragment prepared by the above-mentioned method may be used for the development of P. aeruginosa vaccine or P. aeruginosa infectious drugs, and the like.

The disclosure further provides a vaccine product, including the above-mentioned compound of the P. aeruginosa serotype O5 O-antigen trisaccharide assembled with the amino linker arm.

The disclosure further provides a pharmaceutical composition for treating P. aeruginosa infections, including the above-mentioned compound of the P. aeruginosa serotype O5 O-antigen trisaccharide assembled with the amino linker arm, and a pharmaceutical aid.

In an embodiment of the disclosure, the pharmaceutical aid includes a pharmaceutically acceptable diluent, an adjuvant, a carrier, or an excipient.

### Beneficial Effects

The disclosure proposes for the first time a chemical synthesis method for P. aeruginosa serotype O5 O-antigen trisaccharide. According to the method in the disclosure, the O-antigen trisaccharide is constructed with a D-glucuronic acid building block and a D-fucosamine building block, where the stereoselective synthesis of a 1,2-α-cis-glycosidic bond of D-fucosamine depends on the remote acyl participation and reagent regulation, and the synthesis of two types of 1,2-β-trans-glycosidic bonds of 2,3-diaminomannuronic acids is achieved via S_{N}2 nucleophilic substitution of the azido at position C2; via selective assembly of protecting groups, orthogonal modification of modifying groups, and regulation of the reactivity of glycosyl donors and acceptors, multifunctional modified O-antigen target trisaccharide and its oligosaccharide mimetics are successfully prepared; and a synthesized O-antigen oligosaccharide fragment is immobilized to the surface of glass slide to prepare a glycan microarray, and effective antigens are screened with the serum of a patient to determine the epitope of O-antigen oligosaccharide. The raw materials are cheap and easily available, and the preparation method is simple and easy to repeat. Therefore, the disclosure has a good application prospect in developing vaccines against the P. aeruginosa.

### BRIEF DESCRIPTION OF FIGURES

FIG. 1 shows a trisaccharide repeating unit of a P. aeruginosa serotype O5 O-antigen.

FIG. 2 shows compounds A, B, and C represented by general formulas I, II and III.

FIG. 3 shows compound F represented by general formula VI.

FIG. 4 shows synthesis of sugar building block 9.

FIG. 5 shows synthesis of sugar building block 16.

FIG. 6 shows synthesis of sugar building block 19.

FIG. 7 shows synthesis of trisaccharide 20.

FIG. 8 shows synthesis of target trisaccharide 30.

FIG. 9 shows a synthesis attempt of trisaccharide 35.

FIG. 10 shows a synthesis attempt of trisaccharide 38.

FIG. 11 shows a synthesis attempt of trisaccharide 43 and 44.

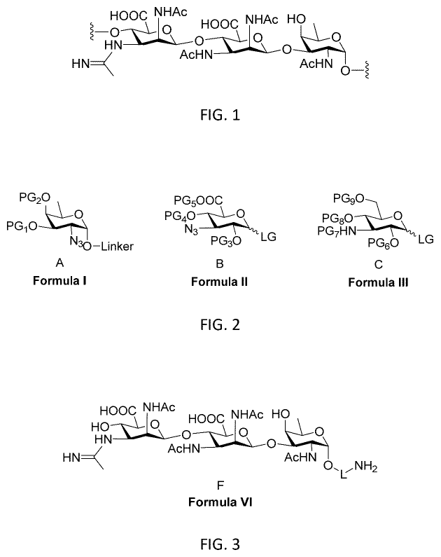

### DETAILED DESCRIPTION

The embodiments of the disclosure will be described in detail below in conjunction with the examples, but those skilled in the art will understand that the following examples are only used to illustrate the disclosure and should not be regarded as limiting the scope of the disclosure. In a case where specific conditions are not specified in the examples, the conventional conditions or the conditions suggested by the manufacturer shall be followed. The used reagents or instruments without manufacturer indicated are all commercially available conventional products.

The yield calculation method in the disclosure is “product (mol)/reaction substrate (mol)*100%”. In the disclosure, structure identification methods for compounds include nuclear magnetic resonance spectroscopy (400 MHZ, 600 MHz), high-resolution mass spectrometry, optical rotation, and infrared spectroscopy. The results are listed in specific synthesis of each compound.

### EXAMPLE 1

### Synthesis of Sugar Building Block 9

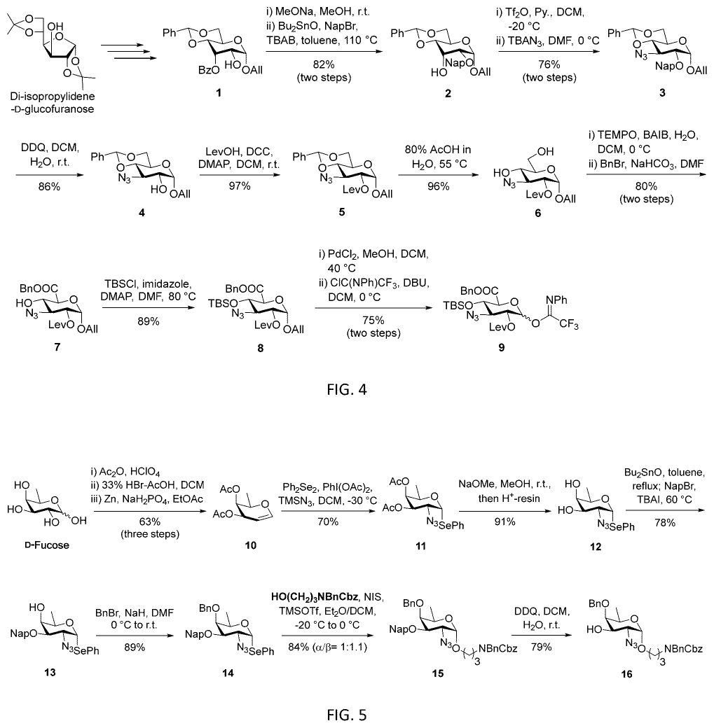

As shown in FIG. 4, taking allyl α-D-allose 1 as a raw material, benzoyl (Bz) was removed with sodium methoxide to afford corresponding diol. Then, through a tin-mediated regioselective 2-naphthylmethylation reaction, naphthylmethylene (Nap) was assembled at 2—OH to afford compound 2. Subsequently, trifluoroacetylation was carried out on O3 in the compound 2, followed by an azide substitution to afford compound 3 with azido at position C3.2-O-Nap was removed from compound 13 with 2,3-dichloro-5,6-dicyano-1,4-benzoquinone (DDQ), and the compound was protected with a levulinyl group to afford compound 5. Subsequently, 4,6-O-benzylidene was hydrolyzed with an 80% acetic acid solution to afford diol 6. Subsequently, hydroxyl at position C6 was oxidized with TEMPO/BAIB, and benzyl esterification of carboxylic acid was carried out to afford compound 7. A temporary protecting group tert-butyldimethylsilyl (TBS) was assembled to the hydroxyl at position C4 to afford compound 8. Finally, terminal allyl was hydrolyzed and converted into trifluoroacetimidate donor 9.

### Specific Experimental Procedures and Steps

Compound 2: compound 1 (6.6 g, 16.01 mmol) was placed in a solution of methanol (40 mL) and dissolved with stirring, and NaOMe (0.43 g, 8.01 mmol) was added. After stirring at room temperature for 5 h, the solution was neutralized with Amberlite IR 120 (H⁺) ion exchange resin and filtered. The filtrate was concentrated under reduced pressure to afford a crude product. The crude product was separated and purified by silica gel column chromatography (PE/EA v/v 2:1) to afford a corresponding diol compound. The above-mentioned compound was subjected to azeotropism with anhydrous toluene, and drying was carried out under high vacuum for 30 min. Then, under nitrogen protection, anhydrous toluene (80 mL) was added, and then Bu₂SnO (5.98 g, 24.02 mmol) and a 4 Å molecular sieve (flame dried) were added. The reaction was refluxed at 110° C., and fully stirred for 3 h. The reaction was cooled to room temperature, 2-bromomethyl-naphthalene (5.32 g, 24.02 mmol) and TBAB (7.74 g, 24.02 mmol) were added and stirred at 60° C. for 5 h. The progress of the reaction was shown by TLC monitoring. After the reaction ended, the reaction mixture was filtered and concentrated under reduced pressure to afford a crude product. The crude product was separated and purified by silica gel column chromatography (PE/EA v/v 6:1) to afford syrup-like compound 2 (5.88 g, 13.13 mmol, 82%). [α]²⁵_{D}=+105.1° (c=1.00, CHCl₃); ¹H NMR (400 MHZ, Chloroform-d) δ7.90-7.30 (m, 12H, Ar-H), 5.95 (dddd, J=17.0, 10.4, 6.3, 5.2 Hz, 1H, All-CH), 5.50 (s, 1H, ArCH), 5.37 (dq, J=17.2, 1.5 Hz, 1H, All-CH₂), 5.25 (dq, J=10.4, 1.3 Hz, 1H, All-CH₂), 4.97-4.88 (m, 2H, 1-H, ArCH), 4.77 (d, J=12.5 Hz, 1H, ArCH), 4.47 (dt, J=6.3, 2.8 Hz, 1H, 3-H), 4.32 (dd, J=10.2, 5.1 Hz, 1H, 6′-H), 4.29-4.16 (m, 2H, All-CH₂, 5-H), 4.07 (ddt, J=12.9, 6.3, 1.3 Hz, 1H, All-CH₂), 3.67 (t, J=10.2 Hz, 1H, 6-H), 3.53 (t, J=3.4 Hz, 1H, 2-H), 3.39 (dd, J=9.7, 2.6 Hz, 1H, 4-H), 3.31 (d, J=7.0 Hz, 1H, 3-OH); ¹³C NMR (101 MHZ, Chloroform-d) δ137.13, 134.75, 133.26, 133.15, 129.05, 128.50, 128.20, 127.86, 127.74, 126.90, 126.32, 126.17, 125.71, 118.39, 102.00, 97.44, 78.93, 73.68, 70.47, 69.14, 69.11, 67.14, 58.09; IR νₘₐₓ (film) 3510, 2929, 1601, 1451, 1316, 1270, 1177, 1106, 1070, 1028, 862, 756, 713 cm⁻¹; HR-ESI-MS (m/z): calcd for C₂₇H₂₈O₆Na⁺ (M+Na⁺): 471.1778, found: 471.1783.

Compound 3: under a nitrogen atmosphere, the compound 2 (5.80 g, 12.94 mmol) was placed in anhydrous DCM (65 mL) and dissolved with stirring. Pyridine (10.6 mL, 129.4 mmol, 10 eq) was added at −20° C. Tf₂O (4.35 mL, 25.88 mmol) was added dropwise, a solvent was stirred, and its temperature was raised from −20° C. to 0° C. within 4 h. After an organic phase was dried over Na₂SO₄, the dried organic phase was concentrated at 30° C. under vacuum to afford a yellow syrup-like chlorate product. Under the nitrogen atmosphere, the above-mentioned chlorate was dissolved in a solution of anhydrous DMF (65 mL), TBAN₃ (11.04 g, 38.82 mmol) was added at 0° C., and the mixture was fully stirred overnight at room temperature. The progress of the reaction was monitored by TLC analysis. After the reaction ended, the reaction solution was diluted with EA (150 mL) and washed with water and brine. After an organic phase was dried over anhydrous Na₂SO₄, the dried organic phase was concentrated under reduced pressure to afford a crude product. The crude product was separated and purified by silica gel column chromatography (PE/EA v/v 10:1) to afford product 3 (4.65 g, 9.83 mmol, yield over two steps 76%). [α]²⁵_{D}=+173.0° (c=1.00, CHCl₃); ¹H NMR (400 MHZ, Chloroform-d) δ7.89-7.34 (m, 12H, Ar-H), 5.92 (dddd, J=17.1, 10.3, 6.6, 5.2 Hz, 1H, All-CH), 5.52 (s, 1H, ArCH), 5.33 (dd, J=17.2, 1.6 Hz, 1H, All-CH₂), 5.23 (dd, J=10.3, 1.4 Hz, 1H, All-CH₂), 4.96 (d, J=12.2 Hz, 1H, ArCH), 4.82 (d, J=12.2 Hz, 1H, ArCH), 4.77 (d, J=3.6 Hz, 1H, 1-H), 4.24 (dd, J=10.3, 4.9 Hz, 1H, 6′-H), 4.18 (ddt, J=12.9, 5.2, 1.5 Hz, 1H, All-CH₂), 4.09 (t, J=9.9 Hz, 1H, 3-H), 3.98 (ddt, J=12.8, 6.6, 1.3 Hz, 1H, All-CH₂), 3.89 (td, J=10.0, 4.9 Hz, 1H, 5-H), 3.65 (t, J=10.3 Hz, 1H, 6-H), 3.47 (dd, J=9.9, 3.6 Hz, 1H, 2-H), 3.39 (t, J=9.8 Hz, 1H, 4-H); ¹³C NMR (101 MHZ, Chloroform-d) δ136.81, 134.89, 133.28, 133.18, 129.13, 128.44, 128.32, 127.93, 127.74, 127.11, 126.29, 126.19, 126.07, 125.92, 118.59, 101.61, 95.80, 79.89, 78.16, 73.34, 68.89, 68.60, 62.68, 61.74; IR νₘₐₓ (film) 2868, 2109, 1451, 1374, 1268, 1094, 1071, 1044, 1028, 995, 929, 751, 713, 699 cm¹; HR-ESI-MS (m/z): calcd for C₂₇H₂₇N₃O₅Na⁺ (M+Na⁺): 496.1843, found: 496.1848.

Compound 4: compound 3 (4.62 g, 9.76 mmol) was dissolved in a solution of DCM (90 mL) and H₂O (10 mL), and DDQ (3.33 g, 14.64 mmol) was added. Stirring was carried out at room temperature for 6.5 h. The progress of the reaction was monitored by TLC analysis. A 5% (w/v) Na₂S₂O₃ solution was added dropwise for quenching. An organic phase was washed with a saturated NaHCO₃ solution and brine, and reverse extraction was carried out with DCM. Subsequently, the organic phase was dried over anhydrous Na₂SO₄ and concentrated under vacuum. The crude product was separated and purified by silica gel column chromatography (PE/EA v/v 4:1) to afford compound 4 (2.79 g, 8.39 mmol, 86%). [α]²⁵_{D}=+162.1° (c 1.00, CH₃Cl). ¹H NMR (400 MHZ, Chloroform-d) δ7.55-7.32 (m, 5H, Ar-H), 5.93 (dddd, J=16.9, 10.3, 6.4, 5.4 Hz, 1H, All-CH), 5.56 (s, 1H, ArCH), 5.34 (dq, J=17.2, 1.5 Hz, 1H, All-CH₂), 5.27 (dq, J=10.3, 1.2 Hz, 1H, All-CH₂), 4.94 (d, J=3.9 Hz, 1H, 1-H), 4.30 (dd, J=10.3, 4.9 Hz, 1H, 6′-H), 4.29-4.23 (m, 1H, All-CH₂), 4.08 (ddt, J=12.6, 6.3, 1.3 Hz, 1H, All-CH₂), 3.89 (td, J=9.9, 4.9 Hz, 1H, 5-H), 3.84 (t, J=9.8 Hz, 1H, 3-H), 3.73 (t, J=10.3 Hz, 1H, 6-H), 3.61 (td, J=10.0, 3.9 Hz, 1H, 2-H), 3.48 (t, J=9.7 Hz, 1H, 4-H), 2.26 (d, J=10.4 Hz, 1H, 2-OH); ¹³C NMR (101 MHZ, Chloroform-d) δ136.78, 132.98, 129.12, 128.32, 126.01, 118.68, 101.53, 97.37, 79.63, 77.22, 71.78, 68.97, 68.85, 63.74, 63.01; IR νₘₐₓ (film) 3345, 2916, 1870, 2105, 1368, 1260, 1152, 1077, 1058, 1013, 994, 931, 746,698, 653 cm⁻¹; HR-ESI-MS (m/z): calcd for C₁₆H₁₉N₃O₅Na⁺ (M+Na⁺): 356.1217, found: 356.1218.

Compound 5: compound 4 (2.77 g, 8.32 mmol) was dissolved in a solution of dried DCM (40 mL), and levulinic acid (LevOH) (1.45 g, 12.48 mmol), N,N-dicyclohexylcarbodiimide (DCC) (2.57 g, 12.48 mmol) and DMAP (1.52 g, 12.48 mmol) were added. Stirring was carried out at room temperature for 4 h until the raw materials were completely depleted, and then DCM was added to dilute the reaction solution. An organic phase was washed with a saturated NaHCO₃ solution and a saturated NaCl solution, and reverse extraction was carried out with DCM. Subsequently, the organic phase was dried over anhydrous Na₂SO₄ and concentrated under vacuum. The crude product was separated and purified by silica gel column chromatography (PE/EA v/v 8:1) to afford compound 5 (3.48 g, 8.07 mmol, 97%). [α]²⁵_{D}=+110.8° (c 1.00, CH₃Cl); ¹H NMR (400 MHz, Chloroform-d) δ7.54-7.33 (m, 5H, Ar-H), 5.91 (dddd, J=16.9, 10.3, 6.2, 5.3 Hz, 1H, All-CH), 5.58 (s, 1H, ArCH), 5.32 (dq, J=17.1, 1.6 Hz, 1H, All-CH₂), 5.24 (dq, J=10.4, 1.4 Hz, 1H, All-CH₂), 5.05 (d, J=3.7 Hz, 1H, 1-H), 4.74 (dd, J=10.3, 3.7 Hz, 1H, 2-H), 4.29 (dd, J=10.4, 4.9 Hz, 1H, 6′-H), 4.21 (ddt, J=12.9, 5.3, 1.5 Hz, 1H, All-CH₂), 4.12 (t, J=10.1 Hz, 1H, 3-H), 4.04 (ddt, J=12.9, 6.2, 1.4 Hz, 1H, All-CH₂), 3.95 (td, J=9.9, 4.9 Hz, 1H, 5-H), 3.75 (t, J=10.3 Hz, 1H, 6-H), 3.54 (t, J=9.7 Hz, 1H, 4-H), 2.92-2.56 (m, 4H, CH₂-Lev), 2.20 (s, 3H, CH₃CO); ¹³C NMR (101 MHZ, Chloroform-d) δ206.02, 171.88, 136.72, 133.23, 129.15, 128.32, 126.03, 118.19, 101.63, 95.20, 79.83, 72.15, 68.97, 68.82, 62.79, 60.04, 37.79, 29.79, 27.88; IR νₘₐₓ (film) 2923, 2868, 2109, 1743, 1718, 1454, 1368, 1260, 1208, 1151, 1123, 1096, 1072, 1045, 996, 933, 753, 700, 653 cm⁻¹; HR-ESI-MS (m/z): calcd for C₂₁H₂₅N₃O₇Na⁺ (M+Na⁺): 454.1585, found: 454.1581.

Compound 6: compound 5 (3.46 g, 8.02 mmol) was dissolved in a solution of 80% AcOH (40 mL). The system was heated with an oil bath to 55° C. and stirring was carried out. The progress of the reaction was monitored by TLC analysis until the raw materials were depleted. After most of the solvent was removed by concentration under reduced pressure, separation and purification by silica gel column chromatography (DCM/MeOH v/v 30:1) were carried out to afford product 6 (2.64 g, 7.70 mmol, 96%). [α]²⁵_{D}=+96.6° (c 1.00, CH₃Cl); ¹H NMR (400 MHZ, Chloroform-d) δ5.90 (dddd, J=17.2, 10.4, 6.1, 5.3 Hz, 1H, All-CH), 5.31 (dq, J=17.2, 1.6 Hz, 1H, All-CH₂), 5.23 (dq, J=10.4, 1.4 Hz, 1H, All-CH₂), 5.03 (d, J=3.6 Hz, 1H, 1-H), 4.69 (dd, J=10.5, 3.6 Hz, 1H, 2-H), 4.19 (ddt, J=13.0, 5.3, 1.5 Hz, 1H, All-CH₂), 4.02 (ddt, J=13.0, 6.1, 1.4 Hz, 1H, All-CH₂), 3.94 (dd, J=10.6, 9.5 Hz, 1H, 3-H), 3.88-3.80 (m, 2H, 6-H), 3.73 (dt, J=9.8, 3.5 Hz, 1H, 5-H), 3.62-3.52 (m, 1H, 4-H), 3.34 (s, 1H, 4-OH), 2.84-2.62 (m, 4H, CH₂-Lev), 2.37 (br, 1H, 6-OH), 2.20 (s, 3H, CH₃CO); ¹³C NMR (101 MHZ, Chloroform-d) δ206.30, 172.03, 133.35, 118.03, 94.57, 72.29, 70.98, 69.06, 68.76, 63.65, 61.78, 37.80, 29.79, 27.90; IR νₘₐₓ (film) 3395, 2925, 2108, 1743, 1716, 1417, 1362, 1258, 1207, 1155, 1040, 929, 840, 770, 607 cm⁻¹; HR-ESI-MS (m/z): calcd for C₁₄H₂₁N₃O₇Na⁺ (M+Na⁺): 366.1272, found: 366.1275.

Compound 7: under argon protection, the compound 6 (2.64 g, 7.70 mmol) was dissolved in a solution of DCM (77 mL) and H₂O (15 mL). The system was cooled to 0° C. with an ice bath, and 2,2,6,6-tetramethylpiperidide oxide (TEMPO) (240 mg, 1.54 mmol) and iodobenzene diacetate (BAIB) (4.96 g, 15.4 mmol) were added. Stirring was carried out at room temperature for 4 h. The progress of the reaction was analyzed by TLC monitoring until the raw materials were depleted. A 5% (w/v) Na₂S₂O₃ solution was added dropwise for quenching. An organic phase was washed with a saturated NaHCO₃ solution and a saturated NaCl solution, and reverse extraction was carried out with DCM. Subsequently, the organic phase was dried over anhydrous Na₂SO₄ and concentrated under vacuum. The crude product was dissolved in anhydrous DMF (77 mL) under nitrogen protection. At room temperature, NaHCO₃ (3.23 g, 38.5 mmol) and BnBr (1.83 mL, 15.4 mmol) were added. When TLC showed that the raw materials were completely depleted, the solvent was removed by vacuum concentration. Purification by silica gel column chromatography (PE/EA v/v 5:1) was carried out. A two-step reaction was carried out to afford glucuronic acid syrup 7 (2.75 g, 6.16 mmol, 80%). [α]²⁵_{D}=+74.6° (c 1.00, CH₃Cl); ¹H NMR (400 MHZ, Chloroform-d) δ7.41-7.32 (m, 5H, Ar-H), 5.90 (dddd, J=17.0, 10.4, 6.2, 5.3 Hz, 1H, All-CH), 5.31 (dq, J=17.2, 1.6 Hz, 1H, All-CH₂), 5.29 (d, J=12.3 Hz, 1H, ArCH), 5.24 (d, J=12.2 Hz, 1H, ArCH), 5.22 (dq, J=10.4, 1.3 Hz, 1H, All-CH₂), 5.10 (d, J=3.6 Hz, 1H, 1-H), 4.68 (dd, J=10.6, 3.6 Hz, 1H, 2-H), 4.27 (d, J=9.8 Hz, 1H, 5-H), 4.25-4.19 (m, 1H, All-CH₂), 4.07 (ddt, J=12.9, 6.2, 1.4 Hz, 1H, All-CH₂), 3.95 (dd, J=10.6, 9.5 Hz, 1H, 3-H), 3.77 (td, J=9.7, 2.8 Hz, 1H, 4-H), 3.17 (s, 1H, 4-OH), 2.87-2.55 (m, 4H, CH₂-Lev), 2.19 (s, 3H, CH₃CO). ¹³C NMR (400 MHZ, Chloroform-d) δ206.00, 171.80, 169.73, 134.85, 133.03, 128.74, 128.67, 128.24, 118.41, 94.86, 71.26, 70.78, 70.17, 69.35, 67.61, 62.45, 37.79, 29.76, 27.84; IR νₘₐₓ (film) 3480, 2935, 2110, 1745, 1717, 1361, 1259, 1181, 1155, 1051, 939, 754, 698, 609 cm⁻¹; HR-ESI-MS (m/z): calcd for C₂₁H₂₅N₃O₈Na⁺ (M+Na⁺): 470.1534, found: 470.1537.

Compound 8: anhydrous DMF (3.5 mL) was added to the glucuronic acid 7 (1.55 g, 3.47 mmol), and the reaction solution was fully stirred under argon protection. Subsequently, tert-butyldimethylsilyl chloride (1.05 g, 6.94 mmol), imidazole (472 mg, 6.94 mmol), and 4-dimethylaminopyridine (43 mg, 0.35 mmol) were added to a reaction flask. Stirring was carried out overnight at 80° C. The progress of the reaction was analyzed by TLC monitoring until the raw materials were depleted. Then, methanol (3 ml) was added to quench the reaction. The reaction solution was diluted with ethyl acetate (50 ml). An organic phase was washed with a saturated NaHCO₃ solution and a saturated NaCl solution, and reverse extraction was carried out with DCM. Subsequently, the organic phase was dried over anhydrous Na₂SO₄ and concentrated under vacuum. The crude product was separated and purified by silica gel column chromatography (PE/EA v/v 10:1) to afford colorless syrup 8 (1.73 g, 3.09 mmol, 89%). [α]²⁵_{D}=+74.5° (c 1.00, CH₃Cl); ¹H NMR (600 MHZ, Chloroform-d) δ7.40-7.32 (m, 5H, Ar-H), 5.90 (dddd, J=16.8, 10.2, 6.3, 5.3 Hz, 1H, All-CH), 5.30 (dd, J=17.2, 1.6 Hz, 1H, All-CH₂), 5.23 (d, J=12.3 Hz, 1H, ArCH), 5.25-5.20 (m, 1H, All-CH₂), 5.13 (d, J=12.4 Hz, 1H, ArCH), 5.07 (d, J=3.6 Hz, 1H, 1-H), 4.75 (dd, J=10.3, 3.6 Hz, 1H, 2-H), 4.23-4.20 (m, 1H, All-CH₂), 4.20 (d, J=9.4 Hz, 1H, 5-H), 4.03 (ddt, J=13.0, 6.3, 1.3 Hz, 1H, All-CH₂), 3.82 (t, J=9.8 Hz, 1H, 3-H), 3.76 (t, J=9.3 Hz, 1H, 4-H), 2.84-2.73 (m, 2H, CH₂-Lev), 2.71-2.60 (m, 2H, CH₂-Lev), 2.19 (s, 3H, CH₃CO), 0.85 (s, 9H, SiC(CH₃)₃), 0.15 (s, 3H, SiCH₃), 0.01 (s, 3H, SiCH₃); ¹³C NMR (151 MHZ, CDCl₃) δ205.90, 171.79, 168.70, 134.98, 133.17, 128.63, 128.49, 128.26, 118.43, 94.88, 72.21, 72.18, 70.94, 69.27, 67.38, 64.49, 37.80, 29.74, 27.88, 25.72, 18.05, −4.43, −5.02; IR νₘₐₓ (film)=2930, 2857, 2109, 1748, 1721, 1456, 1404, 1361, 1252, 1182, 1136, 1052, 939, 838, 781, 749, 697, 669 cm⁻¹; HR-ESI-MS (m/z): calcd for C₂₇H₃₉N₃O₈SiNa⁺ (M+Na⁺): 584.2399, found: 584.2401.

Compound 9: PdCl₂ (485 mg, 2.74 mmol) was added to a solution of MeOH/DCM (v/v, 2/1, 30 mL) of the compound 8 (1.54 g, 2.74 mmol), and the reaction with stirring was carried out at 40° C. for 6 h. After being filtered, the mixture was concentrated under vacuum, and separated and purified by silica gel column chromatography (PE/EA v/v 2:1) to afford corresponding hemiacetal as light yellow syrup. This light yellow syrup was dissolved in a solution of DCM (30 mL). At 0° C., 2,2,2-trifluoro-N-phenylacetimidoyl chloride (2.05 mL, 13.7 mmol) and DBU (1.23 mL, 8.22 mmol) were added to the reaction solution. The reaction system was heated to room temperature, and then, stirring was carried out for 3 h. The progress of the reaction was analyzed by TLC monitoring until the raw materials were depleted. The reaction solution was concentrated under vacuum. The crude product was separated and purified by silica gel column chromatography (PE/EA v/v 20:1→10:1) to afford light yellow syrup 9 (1.43 g, 2.06 mmol, 75%). [α]²⁵_{D}=+29.9° (c 1.00, CH₃Cl); ¹H NMR (600 MHZ, Chloroform-d) δ7.39-6.77 (m, 10H, Ar-H), 5.28 (d, J=12.3 Hz, 1H, ArCH), 5.20-5.15 (m, 1H, 2-H), 5.13 (d, J=12.3 Hz, 1H, ArCH), 4.10-3.78 (m, 2H, 3-H, 5-H), 3.62-3.44 (m, 1H, 4-H), 2.85-2.79 (m, 2H, CH₂-Lev), 2.70-2.59 (m, 2H, CH₂-Lev), 2.18 (s, 3H, CH₃CO), 0.84 (s, 9H, SiC(CH₃)₃), 0.16 (s, 3H, SiCH₃), 0.01 (s, 3H, SiCH₃); ¹³C NMR (151 MHZ, Chloroform-d) δ205.73, 171.02, 166.98, 142.94, 134.69, 128.78, 128.67, 128.63, 128.34, 124.56, 119.26, 94.44, 77.40, 70.86, 70.45, 67.63, 67.48, 37.86, 29.66, 27.72, 25.64, 17.97, −4.53, −5.09; IR νₘₐₓ (film)=2930, 2857, 2109, 1753, 1720, 1597, 1489, 1405, 1363, 1327, 1257, 1212, 1162, 1137, 1090, 910, 839, 779, 754, 696, 584 cm⁻¹; HR-ESI-MS (m/z): calcd for C₃₂H₃₉F₃N₄O₈Na⁺ (M+Na⁺): 715.2381, found: 715.2377.

### EXAMPLE 2

### Synthesis of Sugar Building Block 16

As shown in FIG. 5, taking D-fucose as a raw material, acetylation, bromination, and elimination reactions were carried out to afford diluted sugar 10. Subsequently, under the action of diphenyl diselenide (Ph2Se2) and trimethylsilyl azide (TMSN₃), an azidation reaction was carried out to afford compound 11 with azido at position C₂. Subsequently, deacetylation was carried out under the action of sodium methoxide and selective 2-naphthylmethylation reaction was carried out on O3 to afford compound 13. The benzylation reaction of O4 was carried out in an N,N-dimethylformamide (DMF) solvent by using benzyl bromide and sodium hydride (NaH) to afford compound 14. Subsequently, donor 14 underwent the glycosylation reaction with N-benzyl-N-carbobenzoxy-3-aminopropanol in diethyl ether/dichloromethane to afford compound 15. Finally, 2-naphthylmethylene (Nap) was removed under the action of DDQ to afford glycosyl acceptor 16.

### Specific Experimental Procedures And Steps

Compound 10: under a nitrogen atmosphere, D-fucose (5.0 g, 30.5 mmol) was dissolved in Ac₂O (25.0 mL, 0.266 mol). The reaction system was cooled to 0° C., and then HClO₄ (50 μL, 0.83 mmol) was added. After stirring at room temperature for 30 min, 100 ml of ice water was added to the reaction solution. Subsequently, an organic phase was washed with a saturated NaHCO₃ solution (3×100 mL) and a saturated NaCl solution (100 mL), and reverse extraction was carried out with DCM (3×50 mL). Subsequently, the organic phase was dried over anhydrous Na₂SO₄ and concentrated under vacuum. An organic layer was concentrated to 15 mL and used directly in the next step.

Under the nitrogen atmosphere, peracetylated glucose was dissolved in dried DCM (15 mL). The reaction system was cooled to 0° C., and HBr-AcOH (33% w/w, 10 mL, 57.8 mmol) was added dropwise for consecutive 30 min. After the system was heated to room temperature, and then stirring was carried out for 7 h, the reaction solution was diluted with DCM (50 mL), washed with ice water (3×100 mL), saturated NaHCO₃ (3×100 mL), and brine (100 mL), and dried over Na₂SO₄. After concentration under high vacuum, the crude product proceeded directly to the next step without further purification.

Under the nitrogen atmosphere, the crude product was dissolved in ethyl acetate (100 mL), and saturated NaH₂PO₄ (50 mL) was added. After the reaction was complete as monitored by TLC, the solution was filtered through Celite, and the filtrate was washed with saturated NaHCO₃ (3×100 mL), and reverse extraction was carried out with ethyl acetate (3×50 mL). Subsequently, an organic phase was dried over Na₂SO₄ and concentrated. The concentrated crude product was separated and purified by silica gel column chromatography (PE/EA v/v 20:1) to afford colorless syrup 10 (4.11 g, 19.2 mmol, 63%). [α]²⁵_{D}=+22.1° (c=1.00, CHCl₃); ¹H NMR (400 MHZ, Chloroform-d) δ6.46 (dd, J=6.3, 1.9 Hz, 1H, 1-H), 5.58 (ddd, J=4.1, 2.1, 1.0 Hz, 1H, 4-H), 5.29 (dt, J=4.7, 1.6 Hz, 1H, 2-H), 4.64 (dt, J=6.4, 2.0 Hz, 1H, 3-H), 4.30-4.12 (m, 1H, 5-H), 2.16 (s, 3H, CH₃CO), 2.02 (s, 3H, CH₃CO), 1.28 (d, J=6.6 Hz, 3H, 6-H); ¹³C NMR (101 MHZ, Chloroform-d) δ170.72, 170.42, 146.12, 98.27, 71.54, 66.30, 65.07, 20.87, 20.71, 16.53; IR νₘₐₓ (film) 1747, 1650, 1372, 1243, 1163, 1091, 1073, 1028, 989, 924, 892, 851, 761 cm⁻¹; HR-ESI-MS (m/z): calcd for C₁₀H₁₄O₅Na⁺ (M+Na⁺): 237.0733, found: 237.0734.

Compound 11: compound 10 (3.7 g, 17.3 mmol) was dissolved in dried DCM (86 mL), and then diphenyl diselenide (5.4 g, 17.3 mmol) was added. Under the argon atmosphere, the solution was cooled to −30° C., and then BAIB (5.6 g, 17.3 mmol) and azidotrimethylsilane (3.98 g, 34.6 mmol) were added. The progress of the reaction was analyzed by TLC monitoring until the raw materials were depleted. Washing was carried out with saturated NaHCO₃ (3×100 mL), and reverse extraction was carried out with DCM (3x50 mL). Subsequently, an organic phase was dried over Na₂SO₄ and concentrated under vacuum. The crude product was separated and purified by silica gel column chromatography (PE/EA v/v 5:1) to afford yellowish syrup-like product 11 (5.0 g, 12.1 mmol, 70%). [α]²⁵_{D}=+204.3° (c 1.00, CH₃Cl); ¹H NMR (400 MHZ, Chloroform-d) δ7.68-7.49 (m, 2H, Ar-H), 7.41-7.13 (m, 3H, Ar-H), 5.95 (d, J=5.4 Hz, 1H, 1-H), 5.32 (dd, J=3.3, 1.2 Hz, 1H, 4-H), 5.13 (dd, J=10.8, 3.2 Hz, 1H, 3-H), 4.57-4.44 (m, 1H, 5-H), 4.24 (dd, J=10.8, 5.4 Hz, 1H, 2-H), 2.17 (s, 3H, CH₃CO), 2.07 (s, 3H, CH₃CO), 1.09 (d, J=6.5 Hz, 3H, 6-H); ¹³C NMR (101 MHZ, Chloroform-d) δ170.44, 169.83, 134.77, 129.29, 128.15, 84.54, 71.74, 70.27, 67.54, 58.88, 20.78, 15.93; IR (film): ν=2110, 1747, 1368, 1233, 1084, 1020, 954, 908, 741, 692 cm⁻¹; HR-ESI-MS (m/z): calcd for C₁₆H₁₉N₃O₅SeNa [M+Na]⁺436.0382, found 436.0385.

Compound 12: compound 11 (4.6 g, 11.1 mmol) was dissolved in methanol (55 mL), and NaOMe (0.30 g, 5.55 mmol) was added. The solution was stirred at room temperature for 5 h. The solution was neutralized with Amberlite IR 120 (H⁺) ion exchange resin and then filtered through a cotton plug. The filtrate was concentrated under vacuum. The crude product was separated and purified by silica gel column chromatography (dichloromethane/methanol v/v 100:1) to afford product 12 (3.32 g, 10.1 mmol, yield 91%). [α]_{D}²⁵=+247.6° (c=1.00, CHCl₃); ₁H NMR (400 MHZ, Methanol-d₄) δ=7.66-7.21 (m, 5H, Ar-H), 5.94 (d, J=5.3 Hz, 1H, 1-H), 4.32 (q, J=6.5 Hz, 1H, 5-H), 4.04 (dd, J=10.0, 5.2 Hz, 1H, 2-H), 3.79-3.71 (m, 2H, 3-H, 4-H), 1.18 (d, J=6.5 Hz, 3H, 6-H); ¹³C NMR (100 MHZ, Methanol-d₄) δ135.88, 130.02, 128.70, 86.91, 72.91, 72.66, 70.62, 62.92, 16.41; IR νₘₐₓ (film) 2106, 1093, 1058, 988, 827, 761, 689, 670, 633 cm⁻¹; HR-ESI-MS (m/z): calcd for C₁₂H₁₅N₃O₃SeNa⁺ (M+Na⁺): 352.0171, found: 352.0176.

Compound 13: glycol 12 (1.76 g, 5.35 mmol) was subjected to azeotropism with anhydrous toluene, and drying was carried out under high vacuum for 30 min. Then, under nitrogen protection, anhydrous toluene (50 mL) was added, and then Bu₂SnO (2.0 g, 8.03 mmol) and a 4 Å molecular sieve (flame dried) were added. The reaction was stirred for 2 h under reflux. The reaction was cooled to room temperature, 2-bromomethyl-naphthalene (1.78 g, 8.03 mmol) and TBAB (2.59 g, 8.03 mmol) were added. Stirring was carried out at 60° C. for 4 h. The reaction solution was filtered and concentrated under reduced pressure to remove the solvent. The crude product was separated and purified by silica gel column chromatography (PE/EA v/v 6:1) to afford white solid compound 13 (1.95 g, 4.16 mmol, 78%). [α]_{D}²⁵=+125.9° (c=1.00, CHCl₃); 1H NMR (400 MHZ, Chloroform-d) δ7.91-7.26 (m, 12H, Ar-H), 5.90 (d, J=5.3 Hz, 1H, 1-H), 4.89 (t, J=11.9 Hz, 2H, ArCH), 4.29 (q, J=6.6 Hz, 1H, 5-H), 4.20 (dd, J=10.1, 5.3 Hz, 1H, 2-H), 3.90 (dd, J=3.3, 1.4 Hz, 1H, 4-H), 3.75 (dd, J=10.2, 3.1 Hz, 1H, 3-H), 1.25 (d, J=6.5 Hz, 3H, 6-H); ¹³C NMR (101 MHz, Chloroform-d) δ134.46, 133.27, 133.24, 129.11, 128.65, 128.55, 128.01, 127.80, 127.79, 127.03, 126.40, 126.32, 125.69, 85.22, 79.24, 72.32, 68.64, 60.34, 29.71, 16.05; IR νₘₐₓ (film) 3500, 3054, 2975, 2925, 2111, 1578, 1509, 1476, 1438, 1346, 1271, 1215, 1163, 1091, 1065, 1021, 999, 936, 857, 817, 759, 670, 692, 630, 573 cm⁻¹; HR-ESI-MS (m/z): calcd for C₂₃H₂₃N₃O₃SeNa⁺ (M+Na⁺): 492.0797, found: 492.0799.

Compound 14: NaH (22 mg, 0.54 mmol, dispersity of 60% in mineral oil) was added to a solution of the compound 13 (127 mg, 0.27 mmol) in anhydrous DMF (3 mL), and stirring was carried out at 0° C. for 10 min. Then BnBr (50 μL, 0.41 mmol) was added to the reaction solution, and stirring was carried out at room temperature for 4 h. After the reaction ended, the mixture was cooled to 0° C., and methanol was added dropwise. Subsequently, the reaction solution was washed with water and brine, and reverse extraction was carried out with DCM. An organic phase was dried over anhydrous Na₂SO₄ and concentrated under vacuum to afford a crude product. The crude product was separated and purified by silica gel column chromatography (PE/EA v/v 20:1) to afford compound 14 (135 mg, 0.24 mmol, 89%). [α]_{D}²⁵=+202.8° (c=1.00, CHCl₃); ¹H NMR (400 MHZ, Chloroform-d) δ7.91-7.24 (m, 17H, Ar-H), 5.94 (d, J=5.3 Hz, 1H, 1-H), 4.93 (m, 3H, ArCH), 4.64 (d, J=11.5 Hz, 1H, ArCH), 4.39 (dd, J=10.3, 5.3 Hz, 1H, 2-H), 4.22 (q, J=6.5 Hz, 1H, 5-H), 3.78 (dd, J=10.4, 2.7 Hz, 1H, 3-H), 3.73 (d, J=2.8 Hz, 1H, 4-H), 1.13 (d, J=6.5 Hz, 3H, 6-H); ¹³C NMR (101 MHZ, Chloroform-d) δ138.14, 134.98, 134.38, 133.31, 133.11, 129.01, 128.72, 128.38, 128.30, 128.12, 127.98, 127.75, 127.64, 126.62, 126.25, 126.10, 125.67, 85.56, 80.65, 75.97, 75.04, 72.67, 69.45, 61.09, 29.70, 16.54; IR νₘₐₓ (film) 3056, 2976, 2882, 2108, 1718, 1602, 1578, 1509, 1496, 1476, 1454, 1438, 1346, 1297, 1269, 1213, 1158, 1102, 1080, 1065, 1022, 999, 970, 895, 856, 816, 739, 691, 671, 631, 596, 566 cm⁻¹; HR-ESI-MS (m/z): calcd for C₃₀H₂₉N₃O₃SeNa⁺ (M+Na⁺): 582.1266, found: 582.1268.

Compound 15: compound 14 (20 mg, 0.034 mmol) was added to anhydrous DCM/Et₂O (v/v 3:1, 1 mL) at −20° C., and stirring was carried out under argon protection. Subsequently, N-benzyl-N-carbobenzoxy-3-aminopropanol (15 mg, 0.051 mmol) and a 4 Å molecular sieve (flame dried) were added. The reaction system was cooled to 0° C., and then NIS (9.2 mg, 0.041 mmol) and TMSOTf (1.27 μL, 0.007 mmol) were added. The reaction was stirred at 0° C. for 3 h until TLC detection showed that the raw materials were completely converted. The reaction solution was diluted with DCM and filtered. An organic phase was washed with 10% (w/v) Na₂S₂O₃, a saturated NaHCO₃ solution and a saturated NaCl solution, and reverse extraction was carried out with DCM. Subsequently, the organic phase was dried over anhydrous Na₂SO₄ and concentrated under vacuum. The crude product was separated and purified by silica gel column chromatography (PE/EA v/v 10:1→5:1) to afford product 15 (20 mg, 0.029 mmol, 84%, α:β=1:1.1).

Compound 16: compound 26 (1.45 g, 2.07 mmol) was dissolved in DCM/H₂O (v/v 9:1, 40 mL), and DDQ (0.71 g, 3.11 mmol) was added. After stirring at room temperature for 6 h, quenching was carried out with a 5% (w/v) Na₂S₂O₃ solution, and then, washing was carried out with saturated NaHCO₃ and brine, and reverse extraction was carried out with DCM. An organic phase was dried over anhydrous Na₂SO₄ and concentrated under vacuum. The crude product was separated and purified by silica gel column chromatography (PE/EA v/v 5:1→4:1) to afford compound 11 (0.92 g, 1.64 mmol, 79%). [α]²⁵_{D}=+108.1° (c 1.00, CH₃Cl); ¹H NMR (400 MHZ, Chloroform-d) δ7.62-7.04 (m, 15H, Ar-H), 5.17 (d, J=5.8 Hz, 2H, ArCH), 4.79 (d, J=11.7 Hz, 1H, ArCH), 4.82-4.72 (m, 1H, 1-H), 4.66 (d, J=11.5 Hz, 1H, ArCH), 4.56 (d, J=15.5 Hz, 1H, ArCH), 4.51-4.40 (m, 1H, ArCH), 4.08-3.73 (m, 2H, 3-H, 5-H), 3.71-3.52 (m, 2H, CH₂-Linker, 4-H), 3.51-3.21 (m, 4H, 2-H, CH₂-Linker), 1.94-1.68 (m, 2H, CH₂-Linker), 1.22 (d, J=6.1 Hz, 3H, 6-H); ¹³C NMR (101 MHZ, Chloroform-d) δ156.72, 156.25, 137.82, 136.73, 128.71, 128.58, 128.24, 128.15, 128.01, 127.90, 127.36, 98.24 (C-1), 80.17, 76.20, 68.59, 67.30, 66.48, 65.81, 60.91, 50.87, 44.68, 43.83, 28.37, 27.87, 16.79; IR νₘₐₓ (film) 3444, 2925, 2106, 1695, 1496, 1496, 1454, 1421, 1359, 1221, 1165, 1127, 1103, 1042, 968, 914, 735, 698 cm⁻¹; HR-ESI-MS (m/z): calcd for C₃₁H₃₆N₄O₆ Na⁺ (M+Na⁺): 583.2527, found: 583.2524.

### EXAMPLE 3

### Optimization of Synthesis of 1,2-cis-α-D-fucosamine Glycoside

As shown in Table 1, in order to improve selectivity of the α-glucosidic bond of D-fucoside, a series of glycosyl donors were attempted under different conditions. Taking TMSOTf as an activator, the reaction efficiency of trifluoroacetimidate donor 14a was studied in diethyl ether/dichloromethane, and the resulting product 15 was slightly improved in selectivity of the glycosidic bond, where the selectivity reached α:β=1.5:1. Subsequently, the donor 14a was glycosylated with N-benzyl-N-carbobenzoxy-3-aminopropanol by using a TMSI-Ph₃PO reagent to afford product 15, and its α-configuration selectivity was significantly improved (α/β=4.2:1). In view of the synergistic strategy of combining the remote acyl participation effect with the reagent effect, the stereoselectivity of 1,2-cis-α-aminoglycoside can be enhanced. Therefore, Ac, Bz, and Lev were each assembled on position O4 of the compound 13 to afford donors 14b, 14c, and 14d, respectively, in order to produce α-D-fucoside under their remote acyl participation effects. Finally, under the synergistic effect of the remote participation and the solvent effect, target glycosides 15a, 15b, and 15c were afforded, and each have high yield and excellent stereoselectivity, with a/B selectivity of 7.5:1, 13.5:1, and 12.1:1. Finally, benzoyl was selected as Bz at position O4 as a temporary protecting group because an a-configuration isomer of the compound 15c was easier to separate and had a higher yield (91%). Finally, the benzoyl of the compound 15c was removed by sodium methoxide, and the benzylation reaction was carried out to afford compound 15.

TABLE 1
Optimization of synthesis of 1,2-cis-α-D-fucosamine glycoside

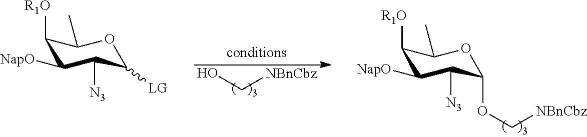

| 14 R₁ = Bn, LG = SePh | | | 15 R₁ = Bn |
|---|---|---|---|
| 14a R₁ = Bn, LG = OC(NPh)CF₃ | | | 15a R₁ = Ac |
| 14b R₁ = Ac, LG = OC(NPh)CF₃ | | | 15b R₁ = Lev |
| 14c R₁ = Lev, LG = OC(NPh)CF₃ 14d R₁ = Bz, LG = OC(NPh)CF₃ | i) MeONa, MeOH, 40° C. ii) BnBr, NaH, DMF, r.t. 82% | [symbol] | 15c R₁ = Bz 15 |

| Sequence | Reaction conditions | Products | α:β (yield %) |
|---|---|---|---|
| 1 | 14, TMSOTf, NIS, DCM, Et₂O, 0° C. | 15 | 1:1.1 (84) |
| 2 | 14a, TMSOTf, DCM, Et₂O, 0° C. | 15 | 1.5:1 (87) |
| 3 | 14a, TMSI, Ph₃PO, DCM, r.t. | 15 | 4.2:1 (91) |
| 4 | 14b, TMSI, Ph₃PO, DCM, r.t. | 15a | 7.5:1 (88) |
| 5 | 14c, TMSI, Ph₃PO, DCM, r.t. | 15b | 13.5:1 (85) |
| 6 | 14d, TMSI, Ph₃PO, DMC, r.t. | 15c | 12.1:1 (91) |

### Specific Experimental Procedures and Steps

Compound 14a: at room temperature, NBS (166 mg, 0.93 mmol) was added to a mixed solution of the compound 14 (130 mg, 0.31 mmol) stirred in THF/H₂O (v/v 4:1, 3 mL), and stirring was carried out for 2.5 h. When TLC showed that the starting materials were completely converted, the mixed solution was diluted with DCM and washed with 10% (w/v) Na₂S₂O₃ and brine. An organic phase was dried over anhydrous Na₂SO_{4,} filtered, and concentrated under reduced pressure. The crude product was separated and purified by silica gel column chromatography (PE/EA v/v 4:1→2:1) to afford corresponding hemiacetal as colorless syrup. This hemiacetal was dissolved in DCM (3 mL). The reaction system was cooled to 0° C., and then 2,2,2-trifluoro-N-phenylacetimidoyl chloride (230 μL, 1.55 mmol) and DBU (140 μL, 0.93 mmol) were added. Subsequently, the reaction system was heated to room temperature, and stirring was carried out for 3 h. The progress of the reaction was analyzed by TLC monitoring until the raw materials were depleted. At room temperature, the reaction solution was concentrated under reduced pressure to afford a crude product. The crude product was separated and purified by silica gel column chromatography (PE/EA v/v 20:1→10:1) to afford light yellow syrup 14a (153 mg, 0.26 mmol, 83%). [α]²⁵_{D}=+33.7° (c 1.00, CH₃Cl); ¹H NMR (400 MHZ, Chloroform-d) δ8.13-6.41 (m, 17H, Ar-H), 4.98 (d, J=11.6 Hz, 1H, ArCH), 4.89 (s, 2H, ArCH), 4.70 (d, J=11.5 Hz, 1H, ArCH), 4.17-3.99 (m, 1H, 5-H), 3.58 (s, 1H, 4-H), 3.56-3.28 (m, 2H, 2-H, 3-H), 1.20 (d, J=6.1 Hz, 3H, 6-H); ¹³C NMR (100 MHZ, Chloroform-d) δ143.43, 137.97, 135.09, 133.25, 133.16, 129.43, 128.70, 128.47, 128.42, 128.36, 127.96, 127.91, 127.79, 126.78, 126.41, 126.36, 126.21, 125.71, 124.29, 120.47, 119.33, 95.97, 80.88, 74.94, 74.68, 72.90, 71.90, 62.17, 29.72, 16.70; IR νₘₐₓ (film) 2931, 2114, 1720, 1598, 1549, 1489, 1452, 1318, 1285, 1211, 1163, 1107, 1082, 1028, 910, 856, 818, 777, 753, 732, 695 cm⁻¹; HR-ESI-MS (m/z): calcd for C₃₂H₂₉F3N₄O₄Na⁺ (M+Na⁺): 613.2033, found: 613.2037.

Compound 15: donor 14a (20 mg, 0.034 mmol) and linker arm N-benzyl-N-carbobenzoxy-3-aminopropanol (15 mg, 0.051 mmol) were subjected to azeotropism with toluene and dried under vacuum overnight. Then, these substances were dissolved in dried DCM/Et₂O (v/v 3:1, 1 mL), and a 4 Å molecular sieve (flame dried) was added. The reaction system is cooled to 0° C. Under argon protection, TMSOTf (1.27 μL, 0.007 mmol) was added to the reaction solution. The reaction solution was stirred at 0° C. until the reaction ended as monitored by TLC. Then triethylamine was added dropwise for quenching, and a 4 Å molecular sieve was filtered and diluted with DCM. An organic phase was washed with saturated NaHCO₃, and then dried over anhydrous Na₂SO₄ and concentrated under vacuum. The crude product was separated and purified by silica gel column chromatography (PE/EA v/v 10:1→5:1) to afford compound 15 (21 mg, 0.03 mmol, 87%, α:β=1.5:1).

The donor 14a (20 mg, 0.034 mmol) and linker N-benzyl-N-carbobenzoxy-3-

aminopropanol (15 mg, 0.051 mmol) were subjected to azeotropism with anhydrous toluene, and then dissolved in dried DCM (1 mL) under argon protection. To a reaction flask were added a 4 Å molecular sieve (flame dried) and Ph₃OP (67 mg, 0.24 mmol). Then TMSI (5 μL, 0.034 mmol, 1.0 eq) was slowly added dropwise into the reaction flask. The reaction was stirred at room temperature until TLC monitoring showed that the reaction ended. The reaction was quenched with saturated Na₂S₂O₃, and a solid 4 Å molecular sieve was filtered off and washed with DCM. An organic phase was washed with water and brine, and dried over anhydrous Na₂SO₄. Subsequently, concentration under vacuum was carried out to afford a crude product. The crude product was separated and purified by silica gel column chromatography (PE/EA v/v 10:1→5:1) to afford compound 15 (22 mg, 0.031 mmol, 91%, α:β=4.2:1).

15α: [α]²⁵_{D}=+74.6° (c 1.00, CH₃Cl). ¹H NMR (600 MHZ, Chloroform-d) δ8.05-7.05 (m, 22H, Ar-H), 5.17 (d, J=9.7 Hz, 2H, ArCH), 4.93 (d, J=11.5 Hz, 1H, ArCH), 4.89-4.75 (m, 3H, ArCH, 1-H), 4.63 (d, J=11.5 Hz, 1H, ArCH), 4.58-4.39 (m, 2H, ArCH), 4.00-3.88 (m, 1H, 4-H), 3.88-3.73 (m, 2H, 2-H, 5-H), 3.72-3.54 (m, 2H, 3-H, CH₂-Linker), 3.51-3.26 (m, 3H, CH₂-Linker), 1.97-1.69 (m, 2H, CH₂-Linker), 1.13 (d, J=7.5 Hz, 3H, 6-H); ¹³C NMR (151 MHZ, Chloroform-d) δ156.70, 156.22, 138.24, 137.87, 136.81, 135.19, 133.32, 133.09, 128.55, 128.49, 128.34, 128.29, 128.26, 127.98, 127.96, 127.85, 127.75, 127.73, 127.31, 126.53, 126.21, 126.03, 125.69, 98.26 (C-1), 77.70, 76.25, 74.94, 72.39, 67.25, 66.69, 65.74, 59.69, 50.85, 50.55, 44.63, 43.81, 28.34, 27.85, 16.75; IR νₘₐₓ (film) 2920, 2107, 1697, 1496, 1454, 1421, 1359, 1218, 1169, 1124, 1044, 965, 914, 857, 818, 749, 699 cm⁻¹; HR-ESI-MS (m/z): calcd for C₄₂H₄₄N₄O₆Na⁺ (M+Na⁺): 723.3153, found: 723.3157.

15β: [α]²⁵_{D}=−30.8° (c 1.00, CH₃Cl). ¹H NMR (400 MHZ, Chloroform-d) δ7.89-7.15 (m, 22H, Ar-H), 5.21-5.10 (m, 2H, ArCH), 4.96 (d, J=11.7 Hz, 1H, ArCH), 4.86 (s, 2H, ArCH), 4.70 (d, J=11.6 Hz, 1H, ArCH), 4.59-4.45 (m, 2H, ArCH), 4.17-4.02 (d, J=8.0 Hz, 1H, 1-H), 3.98-3.84 (m, 1H, CH₂-Linker), 3.83-3.75 (m, 1H, 2-H), 3.54 (dd, J=2.9, 1.0 Hz, 1H, 4-H), 3.52-3.41 (m, 1H, CH₂-Linker), 3.41-3.27 (m, 4H, 3-H, 5-H, CH₂-Linker), 1.96-1.77 (m, 2H, CH₂-Linker), 1.17 (d, J=6.3 Hz, 3H, 6-H); ¹³C NMR (101 MHZ, Chloroform-d) δ156.79, 138.21, 137.96, 135.17, 133.22, 133.08, 128.50, 128.44, 128.35, 128.24, 127.92, 127.88, 127.83, 127.75, 127.72, 127.37, 127.23, 126.64, 126.26, 126.08, 125.75, 102.08 (C-1), 80.68, 75.00, 74.75, 72.69, 70.55, 67.15, 63.20, 50.88, 44.65, 43.60, 28.49, 27.99, 16.84. IR νₘₐₓ (film) 2935, 2110, 1697, 1603, 1496, 1474, 1454, 1420, 1362, 1271, 1218, 1172, 1111, 1067, 1028, 988, 893, 857, 818, 754, 698, 690, 664, 604 cm⁻¹; HR-ESI-MS (m/z): calcd for C₄₂H₄₄N₄O₆Na⁺ (M+Na⁺): 723.3153, found: 723.3156.

Compound 15a: donor 14b (49 mg, 0.09 mmol) and linker arm HO (CH₂)₃NBnCbz (40 mg, 0.136 mmol) were subjected to azeotropism with toluene, and then dissolved in dried DCM (2 mL) under an argon atmosphere. To a reaction flask were added a 4 Å molecular sieve (flame dried) and Ph3OP (200 mg, 0.72 mmol). Then TMSI (13 μL, 0.09 mmol, 1.0 eq) was slowly added dropwise into the reaction flask. The reaction was stirred at room temperature until TLC monitoring showed that the reaction ended. The reaction was quenched with saturated Na₂S₂O₃, and a solid 4 Å molecular sieve was filtered off and washed with DCM. An organic phase was washed with water and brine, and dried over anhydrous Na₂SO₄. Subsequently, concentration under vacuum was carried out to afford a crude product. The crude product was separated and purified by silica gel column chromatography (PE/EA v/v 10:1→5:1) to afford compound 15a (52 mg, 0.079 mmol, 88%, α:β=7.5:1).

Compound 15b: donor 14c (65 mg, 0.10 mmol) and linker arm HO (CH₂)₃NBnCbz (45 mg, 0.15 mmol) were subjected to azeotropism with toluene, and then dissolved in dried DCM (2 mL) under argon. To a reaction flask were added a 4 Å molecular sieve (flame dried) and Ph3OP (222 mg, 0.8 mmol). Then TMSI (15 μL, 0.10 mmol, 1.0 eq) was slowly added dropwise into the reaction flask. The reaction was stirred at room temperature until TLC monitoring showed that the reaction ended. The reaction was quenched with saturated Na₂S₂O₃, and a solid 4 Å molecular sieve was filtered off and washed with DCM. An organic phase was washed with water and brine, and dried over anhydrous Na₂SO_{4.} Subsequently, concentration under vacuum was carried out to afford a crude product. The crude product was separated and purified by silica gel column chromatography (PE/EA v/v 10:1→5:1) to afford compound 15b (60 mg, 0.085 mmol, 85%, α:β=13.5:1).

Compound 15c: donor 14d (1.70 g, 2.81 mmol) and linker arm HO (CH₂)₃NBnCbz (1.26 g, 4.22 mmol) were subjected to azeotropism with toluene, and then dissolved in dried DCM (28 mL) under argon. To a reaction flask were added a 4 Å molecular sieve (flame dried) and Ph₃OP (6.26 g, 22.5 mmol). Then TMSI (0.42 mL, 2.81 mmol, 1.0 eq) was slowly added dropwise into the reaction flask. The reaction was stirred at room temperature until TLC monitoring showed that the reaction ended. The reaction was quenched with saturated Na₂S₂O₃, and a solid 4 Å molecular sieve was filtered off and washed with DCM. An organic phase was washed with water and brine, and dried over anhydrous Na₂SO₄. Subsequently, concentration under vacuum was carried out to afford a crude product. The crude product was separated and purified by silica gel column chromatography (PE/EA v/v 10:1→5:1) to afford compound 15c (1.83 g, 2.56 mmol, 91%, α:β=12.1:1). [α]²⁵_{D}=+159.9° (c 1.00, CH₃Cl); ¹H NMR (400 MHZ, Chloroform-d) δ8.21-7.08 (m, 22H, Ar-H), 5.73-5.59 (m, 1H, 4-H), 5.18 (s, 2H, ArCH), 5.00-4.81 (m, 2H, ArCH, 1-H), 4.73-4.61 (m, 1H, ArCH), 4.59-4.37 (m, 2H, ArCH), 4.16-3.94 (m, 2H, 3-H, 5-H), 3.78-3.58 (m, 2H, 2-H, CH₂-Linker), 3.53-3.27 (m, 3H, CH₂-Linker), 1.85 (s, 2H, CH₂-Linker), 1.18 (d, J=6.1 Hz, 3H, 6-H); ¹³C NMR (101 MHz, Chloroform-d) δ166.14, 156.65, 156.23, 137.77, 136.61, 134.65, 133.29, 133.19, 133.02, 131.46, 129.86, 129.61, 128.55, 128.47, 128.16, 128.00, 127.92, 127.87, 127.60, 127.33, 127.25, 127.01, 125.95, 125.86, 98.19 (C-1), 74.18, 71.40, 69.93, 67.27, 65.97, 65.21, 59.33, 50.84, 50.51, 44.52, 43.71, 28.23, 27.78, 16.33; IR νₘₐₓ (film) 2936, 2108, 1719, 1698, 1601, 1496, 1471, 1452, 1421, 1358, 1314, 1269, 1219, 1169, 1112, 1052, 1026, 968, 857, 819, 752, 712, 670 cm⁻¹; HR-ESI-MS (m/z): calcd for C₄₂H₄₂N₄O₇Na⁺ (M+Na⁺): 737.2946, found: 737.2951 .

Compound 15: NaOMe (70 mg, 1.28 mmol) was added to a solution of the compound 15c (1.82 g, 2.55 mmol) dissolved in methanol (25 mL), and the solution was stirred at 40° C. for 6 h. The solution was neutralized with Amberlite IR 120 (H⁺) ion exchange resin and filtered through a cotton plug. Concentration under reduced pressure was carried out to afford a crude product. Subsequently, the crude product was separated and purified by silica gel column chromatography (PE/EA v/v 5:1→2:1) to afford a corresponding product. The above-mentioned product was dissolved in DMF (13 mL), and NaH (60% dispersion in mineral oil) (205 mg, 5.10 mmol) was added. Then BnBr (0.45 mL, 3.82 mmol) was added at 0° C. The system was heated to room temperature, and then, stirring was carried out at room temperature for 3 h. After the reaction ended as monitored by TLC, the reaction was quenched with methanol, and washed with water and brine, and reverse extraction was carried out with DCM. An organic phase was dried over anhydrous Na₂SO₄ and concentrated under vacuum to afford a crude product. The crude product was separated and purified by silica gel column chromatography (PE/EA v/v 10:1→5:1), and the two-step reaction was carried out to afford compound 15 (1.47 g, 2.1 mmol, 82%). [α]²⁵_{D}=+74.6° (c 1.00, CH₃Cl); ¹H NMR (600 MHZ, Chloroform-d) δ8.05-7.05 (m, 22H, Ar-H), 5.17 (d, J=9.7 Hz, 2H, ArCH), 4.93 (d, J=11.5 Hz, 1H, ArCH), 4.89-4.75 (m, 3H, ArCH, 1-H), 4.63 (d, J=11.5 Hz, 1H, ArCH), 4.58-4.39 (m, 2H, ArCH), 4.00-3.88 (m, 1H, 4-H), 3.88-3.73 (m, 2H, 2-H, 5-H), 3.72-3.54 (m, 2H, 3-H, CH₂-Linker), 3.51-3.26 (m, 3H, CH₂-Linker), 1.97-1.69 (m, 2H, CH₂-Linker), 1.13 (d, J=7.5 Hz, 3H, 6-H); ¹³C NMR (151 MHz, Chloroform-d) δ156.70, 156.22, 138.24, 137.87, 136.81, 135.19, 133.32, 133.09, 128.55, 128.49, 128.34, 128.29, 128.26, 127.98, 127.96, 127.85, 127.75, 127.73, 127.31, 126.53, 126.21, 126.03, 125.69, 98.26 (C-1), 77.70, 76.25, 74.94, 72.39, 67.25, 66.69, 65.74, 59.69, 50.85, 50.55, 44.63, 43.81, 28.34, 27.85, 16.75; IR νₘₐₓ (film) 2920, 2107, 1697, 1496, 1454, 1421, 1359, 1218, 1169, 1124, 1044, 965, 914, 857, 818, 749, 699 cm⁻¹; HR-ESI-MS (m/z): calcd for C₄₂H₄₄N₄O₆Na⁺ (M+Na⁺): 723.3153, found: 723.3157.

### EXAMPLE 4

### Synthesis of Sugar Building Block 19

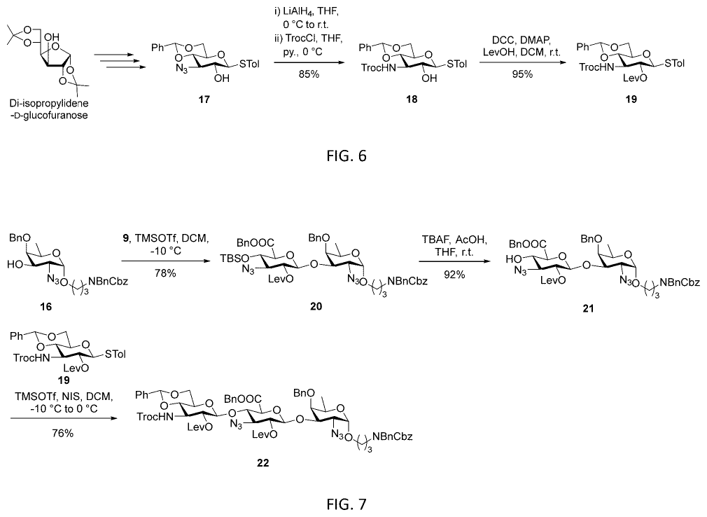

As shown in FIG. 6, taking p-tolylthio-β-D-glucose 17 as a raw material, azido at position C₃ was reduced with lithium tetrahydroaluminum to afford corresponding amino sugar, and then amino at position C₃ was selectively protected with a trichloroethoxycarbonyl (Troc) group to afford compound 18. Subsequently, hydroxyl at position C₂ in the compound 18 was protected with a levulinyl group to afford compound 19.

### Specific Experimental Procedures and Steps

Compound 18: at 0° C., the compound 17 (1.6 g, 4.01 mmol) was placed in a solution of THF (20 mL), and LiAlH₄ (460 mg, 12.03 mmol) was added. The reaction solution was stirred at room temperature for 1.5 h until TLC showed that the raw materials were completely converted. Then, at 0° C., H₂O was added dropwise for quenching, and dilution was carried out with DCM. The reaction solution was washed with H₂O, a saturated NaHCO₃ solution, and brine. An organic phase was dried over anhydrous Na₂SO₄, filtered, and concentrated under vacuum. The crude product was separated and purified by silica gel column chromatography (DCM/MeOHv/v 50:1→20:1) to afford amino sugar. The amino sugar was dissolved in THF (35 mL) and pyridine (0.97 mL, 12.03 mmol, 3 eq). Then at 0° C., a THF (5 mL) mixed solution containing TrocCl (0.55 mL, 4.01 mmol) was added dropwise. The reaction solution was stirred at 0° C. for 2 h until TLC showed that the starting materials were completely converted. Subsequently, methanol was added at 0° C. to quench the reaction. The reaction solution was concentrated under vacuum to afford a crude product. The crude product was separated and purified by silica gel column chromatography (PE/EA v/v 5:1→2:1) to afford colorless syrup-like product 18 (1.86 g, 3.41 mmol, 85%). [α]²⁵_{D}=+57.3° (c 1.00, CH₃Cl); ¹H NMR (400 MHZ, Chloroform-d) δ7.51-7.10 (m, 9H, Ar-H), 5.48 (s, 1H, ArCH), 5.35-5.14 (m, 1H, NH), 4.72 (s, 2H, CH₂-Troc), 4.61 (d, J=9.4 Hz, 1H, 1-H), 4.37 (dd, J=10.6, 4.5 Hz, 1H, 6′-H), 3.85-3.79 (m, 1H, 3-H), 3.75 (t, J=10.2 Hz, 1H, 6-H), 3.63-3.49 (m, 3H, 2-H, 4-H, 5-H), 3.38 (br, 1H, 2-OH), 2.36 (s, 3H, ArCH₃); ¹³C NMR (101 MHZ, Chloroform-d) δ155.26, 138.84, 136.69, 133.71, 129.89, 129.28, 128.32, 127.42, 126.15, 101.75, 95.27, 89.82, 74.69, 71.81, 71.53, 68.58, 58.35, 21.20; IR νₘₐₓ (film) 3328, 2881, 1715, 1542, 1493, 1451, 1372, 1318, 1245, 1168, 1109, 1071, 1031, 1006, 810, 749, 733, 698 cm⁻¹; HR-ESI-MS (m/z): calcd for C₂₃H₂₄Cl₃NO₆SNa⁺ (M+Na⁺): 570.0282, found: 570.0279.

Compound 19: compound 18 (1.84 g, 3.36 mmol) was placed in dried DCM (33 mL) and stirred, and levulinic acid (LevOH) (585 mg, 5.04 mmol), N,N-dicyclohexylcarbodiimide (DCC) (1.04 g, 5.04 mmol) and DMAP (615 mg, 5.04 mmol) were added. The reaction solution was stirred at room temperature for 4 h, until the starting materials were completely depleted, and then the reaction solution was diluted with DCM. An organic phase was washed with a saturated NaHCO₃ solution and brine, dried over anhydrous Na₂SO₄ and filtered, and the filtrate was concentrated under vacuum to afford a crude product. The crude product was separated and purified by silica gel column chromatography (PE/EA v/v 10:1→4:1) to afford compound 19 (2.06 g, 3.19 mmol, 95%). [α]²⁵_{D}=+41.3° (c 1.00, CH₃Cl); ¹H NMR (600 MHZ, Chloroform-d) δ7.46-7.10 (m, 9H, Ar-H), 5.47 (s, 1H, ArCH), 5.12 (d, J=9.0 Hz, 1H, NH), 4.90 (t, J=9.8 Hz, 1H, 2-H), 4.76 (d, J=12.1 Hz, 1H, CH₂-Troc), 4.73 (d, J=9.8 Hz, 1H, 1-H), 4.68 (d, J=12.0 Hz, 1H, CH₂-Troc), 4.37 (dd, J=10.7, 4.4 Hz, 1H, 6′-H), 4.02 (q, J=9.4 Hz, 1H, 3-H), 3.75 (t, J=10.0 Hz, 1H, 6-H), 3.62-3.48 (m, 2H, 4-H, 5-H), 2.83-2.73 (m, 2H, CH₂-Lev), 2.69-2.60 (m, 2H, CH₂-Lev), 2.35 (s, 3H, CH₃CO), 2.19 (s, 3H, ArCH₃); ¹³C NMR (151 MHz, Chloroform-d) δ205.95, 171.82, 154.35, 138.83, 136.74, 133.87, 129.77, 129.14, 128.23, 127.59, 126.13, 101.56, 95.56, 87.07, 77.92, 74.49, 72.00, 70.54, 68.48, 56.75, 37.89, 29.79, 28.01, 21.20; IR νₘₐₓ (film) 3326, 2880, 1742, 1721, 1551, 1375, 1318, 1262, 1245, 1156, 1103, 1069, 1030, 811, 751, 734, 697 cm⁻¹; HR-ESI-MS (m/z): calcd for C₂₈H₃₀Cl₃NO₈SNa⁺ (M+Na⁺): 668.0650, found: 668.0652.

### EXAMPLE 5

### Synthesis of Trisaccharide Intermediate Compound 22

As shown in FIG. 7, taking glycosyl donor 9 and acceptor 16 as raw materials,

glycosylation was carried out under the action of an activating reagent TMSOTf to afford disaccharide compound 20. Subsequently, TBS at position C₄ of disaccharide was removed with tetrabutylammoniumfluoride (TBAF) to afford disaccharide acceptor 21. Finally, monosaccharide donor 19 and disaccharide acceptor 21 were glycosylated under the catalysis of TMSOTf and NIS to afford trisaccharide intermediate compound 22.

### Specific Experimental Procedures and Steps

Compound 20 at −10° C., donor 9 (1.43 g, 2.06 mmol), acceptor 16 (0.77 g, 1.37 mmol) and a 4 Å molecular sieve were dissolved in dried DCM (20 mL). Under argon protection, TMSOTf (75 μL, 0.412 mmol) was added dropwise. When TLC detection showed that the starting materials were completely converted, quenching was carried out with Et₃N, then filtered and concentrated under vacuum. The crude product was separated and purified by silica gel column chromatography (PE/EA v/v 5:1→2:1) to afford colorless syrup 20 (1.71 g, 1.61 mmol, 78%). [α]²⁵_{D}=+24.5° (c 1.00, CH₃Cl); ¹H NMR (400 MHZ, Chloroform-d) δ7.41-7.13 (m, 20H, Ar-H), 5.21-5.12 (m, 4H, ArCH), 5.03-4.97 (m, 1H, 2′-H), 4.83 (d, J=11.4 Hz, 1H, ArCH), 4.81-4.73 (m, 1H, 1-H), 4.72-4.66 (m, 1H, 1′-H), 4.54 (d, J=15.5 Hz, 1H, ArCH), 4.51-4.44 (m, 1H, ArCH), 4.40 (d, J=11.4 Hz, 1H, ArCH), 3.99-3.88 (m, 2H, 5′-H, 3-H), 3.84 (td, J=9.2, 1.3 Hz, 1H, 4′-H), 3.78-3.63 (m, 1H, 5-H), 3.65 (dd, J=10.6, 3.7 Hz, 1H, 2-H), 3.57-3.52 (m, 1H, 4-H), 3.53-3.46 (m, 1H, 3′-H), 3.46-3.36 (m, 1H, CH₂-Linker), 3.38-3.26 (m, 1H, CH₂-Linker), 2.89-2.67 (m, 4H, CH₂-Lev), 2.16 (s, 3H, CH₃CO), 1.93-1.71 (m, 2H, CH₂-Linker), 1.06-0.96 (m, 3H, 6-H), 0.86 (s, 9H, SiC (CH₃)₃), 0.18 (s, 3H, SiCH₃), 0.03 (s, 3H, SiCH₃); ¹³C NMR (151 MHZ, Chloroform-d) δ205.95, 171.33, 167.26, 156.65, 156.26, 138.49, 137.87, 136.77, 134.83, 102.48(C-1′), 98.26 (C-1), 78.96, 78.50, 77.10, 75.11, 71.77, 70.89, 67.75, 67.39, 67.27, 66.46, 65.80, 59.50, 50.89, 50.62, 44.66, 43.89, 37.89, 31.94, 29.71, 29.69, 29.69, 29.68, 27.75, 25.70, 24.76, 22.69, 18.02, 16.34, 14.10, −4.45, −5.01; IR νₘₐₓ (film) 2917, 2850, 2107, 1750, 1697, 1455, 1418, 1362, 1255, 1217, 1134, 1041, 838, 780, 752, 698 cm⁻¹; HR-ESI-MS (m/z): calcd for C₅₅H₆₉N₇O₁₃SiNa⁺ (M+Na⁺): 1086.4615, found: 1086.4619.

Compound 21: at room temperature, the compound 20 (1.70 g, 1.60 mmol) was dissolved in THF (16 mL), then AcOH (91 μL, 1.60 mmol) and TBAF (2.4 mL, 2.4 mmol) were added, and stirring was carried out for 5 h. An organic phase was dried over Na₂SO₄ and concentrated under vacuum. The crude product was separated and purified by silica gel column chromatography (PE/EA v/v 4:1→3:2) to afford colorless syrup 21 (1.40 g, 1.47 mmol, 92%). [α]²⁵_{D}=+21.6° (c 1.00, CH₃Cl). ¹H NMR (600 MHZ, Chloroform-d) δ7.40-7.13 (m, 20H, Ar-H), 5.26-5.11 (m, 4H, ArCH), 4.87 (dd, J=10.3, 7.9 Hz, 1H, 2′-H), 4.80 (d, J=11.4 Hz, 1H, ArCH), 4.79-4.72 (m, 1H, 1-H), 4.72-4.65 (m, 1H, 1′-H), 4.57-4.42 (m, 2H, ArCH), 4.38 (d, J=11.4 Hz, 1H, ArCH), 4.01-3.88 (m, 2H, 5′-H, 3-H), 3.85 (t, J=9.5 Hz, 1H, 4′-H), 3.77-3.64 (m, 1H, 5-H), 3.69-3.63 (m, 2H, 2-H, 4-H), 3.65-3.54 (m, 1H, CH₂-Linker), 3.59 (t, J=9.9 Hz, 1H, 3′-H), 3.51-3.25 (m, 3H, CH₂-Linker), 2.87-2.64 (m, 4H, CH₂-Lev), 2.17 (s, 3H, CH₃CO), 1.91-1.71 (m, 2H, CH₂-Linker), 0.99 (d, J=6.3 Hz, 3H, 6-H); ¹³C NMR (151 MHZ, Chloroform-d) δ206.05, 171.31, 156.23, 138.53, 137.83, 134.45, 128.97, 128.82, 128.58, 128.50, 128.17, 128.02, 127.82, 127.57, 127.38, 127.22, 102.61 (C-1″), 98.24 (C-1), 79.06, 75.14, 74.35, 70.90, 70.81, 67.96, 67.25, 66.47, 65.80, 65.59, 59.30, 50.85, 50.56, 44.67, 43.84, 37.91, 29.75, 27.75, 16.38; IR νₘₐₓ (film) 3445, 2919, 2108, 1750, 1697, 1454, 1422, 1362, 1314, 1218, 1161, 1042, 914, 752, 698 cm⁻¹; HR-ESI-MS (m/z): calcd for C₄₉H₅₅N₇O₁₃Na⁺ (M+Na⁺): 972.3750, found: 972.3756.

Compound 22: at −10° C., the thioglycoside donor 19 (0.56 g, 0.87 mmol), the acceptor 21 (0.55 g, 0.58 mmol) and a 4 Å molecular sieve were placed in dried DCM (6 mL) and stirred, and NIS (235 mg, 1.04 mmol) and TMSOTf (30 μL, 0.174 mmol) were added. The reaction was stirred at 0° C. for 5 h until TLC detection showed that the glycosyl donor was completely converted. The reaction solution was diluted with DCM and filtered. The filtrate was washed with 10% (w/v) Na₂S₂O₃, a saturated NaHCO₃ solution, and brine. An organic phase was dried over anhydrous Na₂SO₄ and filtered, and the filtrate was concentrated under vacuum. The crude product was separated and purified by silica gel column chromatography (PE/EA v/v 5:1→2:1) to afford colorless syrup 22 (648 mg, 0.44 mmol, 76%). [α]²⁵_{D}=+10.2° (c 1.00, CH₃Cl); ¹H NMR (600 MHZ, Chloroform-d) δ7.46-7.16 (m, 25H, Ar-H), 5.47 (s, 1H, ArCH), 5.41 (d, J=11.8 Hz, 1H, ArCH), 5.21-5.14 (m, 2H, ArCH), 5.09 (d, J=11.8 Hz, 1H, ArCH), 4.98 (d, J=9.2 Hz, 1H, NH), 4.87-4.81 (m, 2H, 2′-H, 2″-H), 4.82 (d, J=11.4 Hz, 1H, ArCH), 4.80-4.74 (m, 1H, 1-H), 4.78 (d, J=12.3 Hz, 1H, CH₂-Troc), 4.74-4.67 (m, 2H, 1′-H, CH₂-Troc), 4.54 (d, J=15.5 Hz, 1H, ArCH), 4.52-4.47 (m, 1H, ArCH), 4.46 (d, J=8.1 Hz, 1H, 1″-H), 4.43 (d, J=11.4 Hz, 1H, ArCH), 4.34 (dd, J=10.7, 5.0 Hz, 1H, 6″-H), 4.10-4.03 (m, 2H, 5′-H, 6′-H), 4.00-3.91 (m, 1H, 3-H), 3.88 (q, J=10.3 Hz, 1H, 3″-H), 3.77-3.64 (m, 1H, 5-H), 3.72 (t, J=10.4 Hz, 1H, 6″-H), 3.65 (dd, J=10.8, 3.5 Hz, 1H, 4-H), 3.63-3.51 (m, 4H, 3′-H, CH₂-Linker, 2-H, 4″-H), 3.50-3.29 (m, 3H, CH₂-Linker), 3.18 (td, J=9.6, 5.0 Hz, 1H, 5″-H), 2.86-2.49 (m, 8H, CH₂-Lev), 2.18 (s, 3H, CH₃CO), 2.17 (s, 3H, CH₃CO) 1.92-1.71 (m, 2H, CH₂-Linker), 1.05-0.95 (m, 3H, 6-H); ¹³C NMR (151 MHZ, Chloroform-d) δ206.08, 206.02, 172.10, 171.31, 166.67, 154.37, 138.40, 137.83, 136.75, 134.57, 129.34, 129.17, 129.02, 128.66, 128.65, 128.57, 128.51, 128.26, 128.22, 128.01, 127.83, 127.67, 127.38, 127.24, 126.13, 102.51 (C-1′), 101.60 (ArCH), 100.52 (C-1″), 98.23 (C-1), 95.54 (CCl₃-Troc), 78.81, 78.03, 76.23, 74.98, 74.48, 71.83, 70.46, 68.39, 67.82, 67.59, 67.25, 66.38, 65.79, 64.61, 59.40, 54.95, 50.56, 44.61, 37.91, 37.79, 29.83, 29.75, 27.84, 27.73, 27.63, 16.37; IR νₘₐₓ (film) 3323, 2924, 2111, 1719, 1543, 1455, 1420, 1364, 1314, 1274, 1239, 1152, 1081, 1029, 917, 820, 752, 699 cm⁻¹; HR-ESI-MS (m/z): calcd for C₇₀H₇₇Cl₃N₈O₂₁Na⁺ (M+Na⁺): 1493.4161, found: 1493.4168.

### EXAMPLE 6

### Synthesis of Target Trisaccharide 30

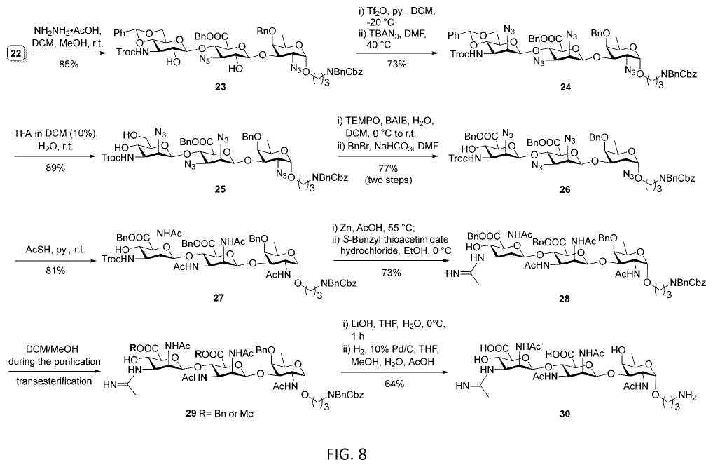

As shown in FIG. 8, taking trisaccharide intermediate compound 22 as a raw material, two levulinyl groups in the compound 22 were simultaneously removed under the action of hydrazine acetate to afford diol 23. Subsequently, two hydroxyl groups in the compound 23 were converted into trifluoromethylsulfonyl by using trifluoroanhydride (Tf₂O) and pyridine, and then replacement was carried out with tetrabutylammonium azide (TBAN₃) at the axial position to afford compound 24. Then, after 4,6-O-benzylidene was hydrolyzed under the mediation of trifluoroacetic acid (TFA), the hydroxyl at position C_{6″} of the resulting diol 25 was selectively oxidized to produce corresponding carboxylic acid, and then selective benzyl esterification reaction was carried out on the carboxylic acid by using benzyl bromide and sodium bicarbonate (NaHCO₃) to afford compound 26. The azido was reduced with thioacetic acid (AcSH) in pyridine to afford acetylamino sugar 27. Subsequently, a Troc group on the trisaccharide 27 was removed in acetic acid with excessive zinc powder at 55° C., and then S-benzyl thioacetimidate hydrochloride was used to react in pyridine to afford trisaccharide 28 containing acetamidino at position C_{3″.} Since the polarity of the trisaccharide 26 was increased, the trisaccharide was difficult to dissolve in dichloromethane and is easy to dissolve in methanol. It was found that during dissolution, transfer, and storage of the trisaccharide in methanol, part of the product 28 underwent ester exchange reaction from benzyl carboxylate to methyl carboxylate due to the action of the alkaline acetamidino to afford trisaccharide 29. Finally, the methyl ester on the trisaccharide 29 was hydrolyzed by lithium hydroxide at 0° C., and then total deprotection was achieved with the Pd/C hydrogenation reaction to afford target trisaccharide 30.

### Specific Experimental Procedures and Steps

Compound 23: compound 22 (620 mg, 0.422 mmol) was placed in DCM/MeOH (v/v 20:1, 4.2 mL) and stirred, and hydrazine acetate (117 mg, 1.27 mmol) was added. The reaction solution was stirred at room temperature for 4.5 h until TLC showed that the starting materials were completely converted. The reaction solution was diluted with DCM, and washed with a saturated NaHCO₃ solution and brine. An organic phase was dried over anhydrous Na₂SO₄ and concentrated under vacuum. The crude product was separated and purified by silica gel column chromatography (PE/EA v/v 5:1→2:1) to afford colorless syrup product 23 (457 mg, 0.359 mmol, 85%). [α]²⁵_{D}=+35.8° (c 1.00, CH₃Cl); ¹H NMR (600 MHZ, Chloroform-d) δ7.49-7.14 (m, 25H, Ar-H), 5.48 (s, 1H, ArCH), 5.29 (d, J=11.9 Hz, 1H, ArCH), 5.23 (s, 1H, NH), 5.19-5.11 (m, 3H, ArCH), 4.86-4.78 (m, 1H, 1-H), 4.77 (d, J=11.7 Hz, 1H, ArCH), 4.77-4.72 (m, 2H, CH₂-Troc), 4.58-4.49 (m, 1H, 1′-H), 4.50 (d, J=7.4 Hz, 1H, 1″-H), 4.43 (d, J=11.6 Hz, 1H, ArCH), 4.40 (dd, J=10.7, 5.0 Hz, 1H, 6″-H), 4.08-4.02 (m, 1H, 5′-H), 4.03-3.94 (m, 1H, 3-H), 3.91 (t, J=9.5 Hz, 1H, 4′-H), 3.80-3.65 (m, 3H, 6″-H, 2-H, 5-H), 3.72 (dd, J=10.1, 7.6 Hz, 1H, 3″-H), 3.65-3.56 (m, 1H, CH₂-Linker), 3.65-3.60 (m, 2H, 4″-H, 4-H), 3.59-3.51 (m, 2H, 2″-H, 3′-H), 3.43-3.39 (m, 1H, 5″-H), 3.48-3.28 (m, 3H, CH₂-Linker), 3.38 (dd, J=9.9, 7.7 Hz, 1H, 2′-H), 2.90 (s, 1H, 2′-OH), 1.91-1.70 (m, 2H, CH₂-Linker), 1.04-0.94 (m, 3H, 6-H); ¹³C NMR (151 MHZ, Chloroform-d) δ167.89, 155.05, 138.35, 137.79, 136.82, 134.56, 129.24, 129.08, 128.85, 128.59, 128.54, 128.51, 128.40, 128.31, 128.24, 128.03, 127.85, 127.74, 127.41, 127.25, 126.17, 105.07 (C-1′), 104.34 (C-1″), 101.78 (ArCH), 97.87 (C-1), 95.39 (CCl₃-Troc), 80.27, 75.02, 74.67, 74.46, 73.11, 72.47, 68.50, 68.16, 67.86, 67.28, 66.88, 66.36, 65.89, 59.35, 56.90, 27.82, 27.23, 16.35. IR νₘₐₓ (film) 3390, 2925, 2112, 1743, 1683, 1537, 1497, 1454, 1423, 1363, 1239, 1220, 1176, 1082, 1029, 916, 819, 752, 698 cm⁻¹; HR-ESI-MS (m/z): calcd for C₆₀H₆₅Cl₃N₈O₁₇Na⁺ (M+Na⁺): 1297.3425, found: 1297.3431.

Compound 24: under nitrogen protection, the compound 23 (440 mg, 0.345 mmol) was placed in a mixed solution of dried DCM (6.0 mL) and pyridine (0.56 mL, 6.9 mmol, 20 eq) and stirred. The reaction system was cooled to −20° C., and then a mixed solution of dried DCM (1 mL) containing Tf₂O (0.35 mL, 2.07 mmol) was added dropwise. The reaction solution was stirred, and its temperature was raised from −20° C. to 0° C. within 4 h. After an organic phase was dried over anhydrous sodium sulfate, the dried organic phase was concentrated at 30° C. under vacuum to afford a yellow syrup-like chlorate product. Under the nitrogen atmosphere, the above-mentioned chlorate was dissolved in a solution of anhydrous DMF (7 mL). TBAN₃ (0.98 g, 3.45 mmol) was added at 0° C. The reaction was stirred overnight at room temperature. The progress of the reaction was analyzed by TLC monitoring. The reaction solution was diluted with ethyl acetate (20 mL), and washed with water and brine. An organic phase was dried over anhydrous Na₂SO₄ and concentrated under vacuum. The crude product was separated and purified by silica gel column chromatography (PE/EA v/v 2:1). A two-step reaction was carried out to afford product 24 (334 mg, 0.252 mmol, 73%). [α]²⁵_{D}=−8.4° (c=1.00, CHCl₃); ¹H NMR (600 MHz, Chloroform-d) δ7.60-7.05 (m, 25H, Ar-H), 5.46 (s, 1H, ArCH), 5.29 (d, J=11.6 Hz, 1H, ArCH), 5.25 (d, J=8.5 Hz, 1H, NH), 5.22-5.12 (m, 3H, ArCH), 4.99 (d, J=11.4 Hz, 1H, ArCH), 4.92-4.81 (m, 1H, 1′-H), 4.81-4.72 (m, 3H, CH₂-Troc, 1-H), 4.66 (s, 1H, 1″-H), 4.59-4.43 (m, 3H, ArCH), 4.35 (dd, J=11.1, 4.9 Hz, 1H, 6″-H), 4.22 (t, J=9.6 Hz, 1H, 4′-H), 4.14-4.08 (m, 1H, 5′-H), 4.08-3.97 (m, 1H, 3-H), 3.96-3.85 (m, 3H, 3″-H, 2-H, 2′-H), 3.83 (s, 1H, 2″-H), 3.78 (t, J=10.4 Hz, 1H, 6″-H), 3.75-3.68 (m, 1H, 5-H), 3.68-3.53 (m, 2H, 4-H, CH₂-Linker), 3.56 (t, J=9.7 Hz, 1H, 4″-H), 3.52-3.26 (m, 4H, CH₂-Linker, 3′-H), 3.22 (td, J=9.9, 4.9 Hz, 1H, 5″-H), 1.90-1.70 (m, 2H, CH₂-Linker), 1.01-0.92 (m, 3H, 6-H); ¹³C NMR (151 MHZ, Chloroform-d) δ167.18, 154.03, 138.34, 137.84, 136.85, 134.67, 129.40, 129.28, 129.18, 129.04, 128.75, 128.70, 128.59, 128.52, 128.27, 128.24, 128.13, 128.02, 127.89, 127.81, 127.75, 127.39, 127.20, 126.14, 102.01 (C-1′), 101.95 (C-1″), 101.40 (ArCH), 97.52 (C-1), 95.38 (CCl₃-Troc), 80.80, 78.25, 75.77, 75.02, 74.66, 74.57, 74.25, 68.36, 68.17, 68.01, 67.23, 66.41, 65.75, 63.57, 62.68, 62.23, 60.40, 59.43, 52.08, 50.77, 50.52, 44.57, 43.72, 28.20, 27.83, 16.39; IR νₘₐₓ (film) 2920, 2108, 1743, 1693, 1455, 1275, 1234, 1178, 1090, 1045, 1000, 905, 822, 753, 699 cm⁻¹; HR-ESI-MS (m/z): calcd for C₆₀H₆₃Cl₃N₁₄O₁₅Na⁺ (M+Na⁺): 1347.3555, found: 1347.3559 .

Compound 25: the compound 24 (240 mg, 0.181 mmol) was placed in a solution of DCM (4.0 mL) and stirred, and trifluoroacetic acid (0.5 mL, 6.72 mmol) and H₂O (33 μL, 1.81 mmol, 10 eq) were added. The reaction was stirred at room temperature for 4 h until TLC showed that the starting materials were completely converted. The reaction solution was diluted with DCM, and washed with water, a saturated NaHCO₃ solution, and brine. An organic phase was dried over anhydrous Na₂SO₄ and concentrated under vacuum. The crude product was separated and purified by silica gel column chromatography (DCM/MeOH v/v 50:1→20:1) to afford colorless syrup 25 (199 mg, 0.161 mmol, 89%). [α]²⁵_{D}=−5.5° (c 1.00, CH₃Cl); ¹H NMR (600 MHZ, Chloroform-d) δ7.42-7.14 (m, 20H, Ar-H), 5.53 (d, J=7.8 Hz, 1H, NH), 5.28-5.11 (m, 4H, ArCH), 4.99 (d, J=11.5 Hz, 1H, ArCH), 4.93-4.79 (m, 1H, 1′-H), 4.82 (d, J=11.9 Hz, 1H, CH₂-Troc), 4.82-4.70 (m, 1H, 1-H), 4.77 (d, J=12.0 Hz, 1H, CH₂-Troc), 4.62 (s, 1H, 1″-H), 4.56 (d, J=11.5 Hz, 1H, ArCH), 4.55-4.42 (m, 2H, ArCH), 4.21 (t, J=9.6 Hz, 1H, 4′-H), 4.09 (d, J=8.8 Hz, 1H, 5′-H), 4.07-3.97 (m, 1H, 3-H), 3.97-3.86 (m, 1H, 2-H), 3.93-3.86 (m, 1H, 2′-H), 3.92 (dd, J=12.2, 3.1 Hz, 1H, 6″-H), 3.83 (dd, J=12.3, 3.9 Hz, 1H, 6″-H), 3.79-3.66 (m, 1H, 5-H), 3.66-3.55(m, 5H, 4″-H, 4-H, CH₂-Linker, 2″-H, 3″-H), 3.53-3.26 (m, 4H, 3′-H, CH₂-Linker), 3.15 (dt, J=8.1, 3.5 Hz, 1H, 5″-H), 2.84 (br, 1H, 4″-OH), 2.28 (br, 1H, 6″-OH), 1.93-1.70 (m, 2H, CH₂-Linker), 1.02-0.91 (m, 3H, 6-H); ¹³C NMR (151 MHZ, Chloroform-d) δ167.20, 156.61, 156.25, 155.16, 138.28, 137.79, 136.57, 134.67, 129.34, 129.25, 128.98, 128.78, 128.59, 128.50, 128.23, 128.02, 127.81, 127.75, 127.42, 127.37, 127.17, 101.86 (C-1′), 101.25 (C-1″), 97.56 (C-1), 95.24 (CCl₃-Troc), 80.71, 78.19, 75.02, 74.92, 74.55, 74.47, 67.97, 67.24, 66.46, 66.38, 65.73, 63.32, 62.42, 62.06, 61.88, 59.40, 54.55, 50.74, 50.49, 44.54, 43.74, 28.16, 27.75, 16.37. IR νₘₐₓ (film) 3411, 2926, 2109, 1738, 1697, 1516, 1455, 1423, 1362, 1273, 1168, 1076, 1045, 820, 737, 699 cm⁻¹; HR-ESI-MS (m/z): calcd for C₅₃H₅₉Cl₃N₁₄O₁₅Na⁺ (M+Na⁺): 1259.3242, found: 1259.3247.

Compound 26: under argon protection, the compound 25 (190 mg, 0.154 mmol) was dissolved in a solution of DCM (4 mL) and H₂O (1 mL). The system was cooled to 0° C. with an ice bath, and then TEMPO (5 mg, 0.031 mmol) and BAIB (99 gm, 0.308 mmol) were added. Stirring was carried out at room temperature for 4 h. The progress of the reaction was analyzed by TLC monitoring until the raw materials were depleted. A 10% (w/v) Na₂S₂O₃ solution was added dropwise for quenching. An organic phase was washed with a saturated NaHCO₃ solution and a saturated NaCl solution, and reverse extraction was carried out with DCM. Subsequently, the organic phase was dried over anhydrous Na₂SO₄ and concentrated under vacuum. The crude product was dissolved in anhydrous DMF (3 mL) under nitrogen protection. At room temperature, NaHCO₃ (65 mg, 0.77 mmol) and BnBr (55 μL, 0.462 mmol) were added. When TLC showed that the raw materials were completely depleted, the solvent was removed by vacuum concentration. Purification by silica gel column chromatography (PE/EA v/v 2:1) was carried out. A two-step reaction was carried out to afford syrup 26 (2.75 g, 6.16 mmol, 80%). [α]²⁵_{D}=−12.5° (c 1.00, CH₃Cl); ¹H NMR (600 MHZ, Chloroform-d) δ7.43-7.13 (m, 25H, Ar-H), 5.36 (d, J=8.4 Hz, 1H, NH), 5.27 (d, J=12.2 Hz, 1H, ArCH), 5.26-5.22 (m, 1H, ArCH), 5.21 (d, J=12.2 Hz, 1H, ArCH), 5.19-5.12 (m, 3H, ArCH), 4.98 (d, J=11.5 Hz, 1H, ArCH), 4.90-4.77 (m, 1H, 1′-H), 4.84 (d, J=12.0 Hz, 1H, CH₂-Troc), 4.80-4.72 (m, 1H, 1-H), 4.72 (d, J=12.0 Hz, 1H, CH₂-Troc), 4.64 (s, 1H, 1″-H), 4.56 (d, J=11.5 Hz, 1H, ArCH), 4.56-4.43 (m, 2H, ArCH), 4.22 (t, J=9.6 Hz, 1H, 4′-H), 4.11-3.96 (m, 2H, 5′-H, 3-H), 3.95-3.83 (m, 2H, 2-H, 2′-H), 3.77-3.61 (m, 6H, 4″-H, 5-H, 4-H, 2″-H, 3″-H, 5″-H), 3.63-3.53 (m, 1H, CH₂-Linker), 3.53-3.27 (m, 4H, 3′-H, CH₂-Linker), 3.17 (br, 1H, 4″-OH), 1.89-1.70 (m, 2H, CH₂-Linker), 0.96 (t, J=8.5 Hz, 3H, 6″-H); ¹³C NMR (151 MHz, Chloroform-d) δ168.67, 167.18, 156.60, 156.22, 154.55, 138.32, 137.80, 136.83, 136.59, 134.71, 129.33, 129.23, 128.97, 128.78, 128.63, 128.58, 128.49, 128.43, 128.21, 128.01, 127.80, 127.72, 127.38, 127.16, 101.73 (C-1′), 101.39 (C-1″), 97.56 (C-1), 95.24 (CCl₃-Troc), 80.65, 78.12, 75.34, 74.96, 74.89, 74.87, 74.48, 67.95, 67.79, 67.22, 66.37, 65.71, 63.35, 62.62, 61.66, 59.39, 53.75, 50.77, 50.49, 44.54, 43.72, 28.17, 27.74, 16.38. IR νₘₐₓ (film) 3367, 2925, 2110, 1743, 1697, 1517, 1455, 1362, 1273, 1176, 1127, 1075, 1044, 1001, 823, 753, 698 cm⁻¹; HR-ESI-MS (m/z): calcd for C₆₀H₆₃Cl₃N₁₄O₁₆Na⁺ (M+Na⁺): 1363.3504, found: 1259.3501.

Compound 27: under nitrogen protection, the compound 26 (56 mg, 0.042 mmol) was placed in a solution of pyridine (2.1 mL) and stirred. The reaction system was cooled to 0° C., and then AcSH (2.1 mL) was added. The reaction was stirred at room temperature until TLC detection showed that the starting materials were completely converted. The reaction solution was diluted with DCM, and washed with water, a saturated NaHCO₃ solution, and brine. An organic phase was dried over anhydrous Na₂SO₄ and concentrated under vacuum. The crude product was separated and purified by silica gel column chromatography (DCM/MeOH v/v 50:1→20:1) to afford colorless syrup product 27 (48 mg, 0.034 mmol, 81%). [α]²⁵_{D}=+16.2° (c 1.00, CH₃Cl); ¹H NMR (600 MHZ, Chloroform-d) δ7.91-7.74 (m, 1H, NH), 7.45-7.16 (m, 25H, Ar-H), 6.96-6.86 (m, 1H, NH), 6.86-6.79 (m, 1H, NH), 6.80-6.72 (m, 1H, NH), 6.08-5.86 (m, 1H, NH), 5.29 (d, J=11.9 Hz, 1H, ArCH), 5.23-5.04 (m, 5H, ArCH), 4.91-4.83 (m, 2H, 1′-H, ArCH), 4.80 (d, J=12.0 Hz, 1H, CH₂-Troc), 4.72 (d, J=12.1 Hz, 1H, CH₂-Troc), 4.71-4.66 (m, 1H, 3′-H), 4.65-4.59 (m, 2H, 1-H, ArCH), 4.54 (d, J=11.8 Hz, 1H, ArCH), 4.38 (d, J=15.9 Hz, 1H, ArCH), 4.31-4.19 (m, 3H, 3″-H,-H, 1″-H), 4.09 (d, J=9.8 Hz, 1H, 5″-H), 3.98 (q, J=10.6, 9.5 Hz, 1H, 4″-H), 3.90-3.82 (m, 2H, 4′-H, 5-H), 3.79-3.71 (m, 3H, 3-H, 4-H, CH₂-Linker), 3.68 (s, 1H, 2′-H), 3.65-3.61 (m, 1H, CH₂-Linker), 3.55 (d, J=9.5 Hz, 1H, 5′-H), 3.33-3.23 (m, 2H, CH₂-Linker, 2-H), 3.23-3.15 (m, 1H, CH₂-Linker), 2.13 (s, 3H, CH₃CO), 2.02 (s, 3H, CH₃CO), 1.89 (s, 3H, CH₃CO), 1.78 (s, 3H, CH₃CO), 1.08 (d, J=6.4 Hz, 3H, 6-H); ¹³C NMR (151 MHZ, Chloroform-d) δ175.35, 172.52, 172.22, 171.43, 168.43, 167.65, 157.71, 156.53, 138.68, 137.33, 136.40, 135.02, 134.75, 129.51, 129.38, 129.00, 128.71, 128.65, 128.55, 128.47, 128.24, 128.15, 127.83, 127.70, 127.61, 127.22, 101.44 (C-1′), 98.02 (C-1″), 97.82 (C-1), 95.26 (CCl₃-Troc), 80.59, 79.40, 74.96, 74.21, 69.58, 67.79, 67.48, 67.33, 66.76, 63.43, 57.96, 52.78, 51.84, 51.22, 49.65, 48.50, 42.66, 27.20, 23.50, 23.22, 23.15, 22.78, 16.64; IR νₘₐₓ (film) 3306, 2926, 1743, 1668, 1537, 1455, 1423, 1373, 1240, 1180, 1093, 1050, 819, 736, 698 cm⁻¹; HR-ESI-MS (m/z): calcd for C₆₈H₇₉Cl₃N₆O₂₅D =+20Na⁺ (M+Na⁺): 1427.4307, found: 1427.4311.

Compound 28: compound 27 (30 mg, 21.4 μL) was placed in a solution of acetic acid (2.0 mL) and stirred, and activated zinc powder (50 mg, 0.78 mmol) was added. After stirring at room temperature for 12 h, the reaction mixture was filtered through Celite, and the filtrate was concentrated under vacuum. The resulting crude product was separated and purified by silica gel column chromatography (DCM/MeOH v/v 20:1→10:1) to afford corresponding amino sugar. At 0° C., the above-mentioned aminosaccharide was placed in a solution of anhydrous pyridine (2 mL) and stirred, and benzyl thioacetimidate hydrochloride (8.6 mg, 42.8 μmol) was added. The reaction was stirred at 0° C. for 5 h. Subsequently, the reaction solution was concentrated under vacuum. The crude product was separated and purified by silica gel column chromatography (DCM/MeOH v/v 10:1). A two-step reaction was carried out to afford colorless syrup product 28 (19 mg, 15.6 μmol, 73%). [α]²⁵_{D}=−2.7° (c 1.00, CH₃Cl); ¹H NMR (600 MHZ, Methanol-d₄) δ7.43-7.17 (m, 25H, Ar-H), 5.37 (d, J=12.3 Hz, 1H, ArCH), 5.26 (s, 2H, ArCH), 5.19-5.10 (m, 3H, ArCH), 5.08 (s, 1H, 1′-H), 4.99-4.90 (m, 1H, 1-H), 4.88 (d, J=11.4 Hz, 1H, ArCH), 4.65-4.58 (m, 1H, 1″-H), 4.57-4.53 (m, 1H, 2-H), 4.53-4.50 (m, 2H,), 4.47-4.45 (m, 1H, 2′-H), 4.45-4.39 (m, 1H, 2″-H), 4.35 (d, J=11.3 Hz, 1H, ArCH), 4.26-4.15 (m, 2H, 3-H, 5-H), 4.13-4.06 (m, 2H, 5′-H, 4-H), 4.01-3.89 (m, 2H, 3″-H, 3′-H), 3.83-3.76 (m, 2H, 5-H, 4′-H), 3.66-3.59 (m, 1H, 4″-H), 3.59-3.48 (m, 1H, CH₂-Linker), 3.46-3.33 (m, 3H, CH₂-Linker), 2.20 (s, 3H, CH₃CO), 2.02 (s, 3H, CH₃CO), 1.95-1.90 (m, 3H, CH₃CO), 1.85 (s, 3H, CH₃CO), 1.81 (s, 3H, CH₃CO), 1.05-0.95 (m, 3H, 6-H); ¹³C NMR (151 MHZ, Methanol-d₄) δ175.03, 174.27, 173.88, 173.71, 170.65, 169.77, 167.49, 140.34, 139.29, 138.10, 136.71, 136.56, 129.77, 129.66, 129.21, 129.17, 128.93, 128.53, 128.33, 127.99, 103.49 (C-1), 100.69 (C-1′), 99.03 (C″-1), 80.53, 78.19, 76.46, 76.14, 68.81, 68.47, 67.73, 67.58, 66.05, 58.19, 53.03, 52.26, 51.35, 50.21, 45.66, 45.49, 29.43, 28.80, 23.04, 22.87, 22.78, 19.24, 16.97; IR νₘₐₓ (film) 3291, 2931, 1743, 1657, 1550, 1454, 1374, 1293, 1244, 1177, 1112, 1051, 739, 699, 599 cm⁻¹; HR-ESI-MS (m/z): calcd for C₆₇H₈₂N₇O₁₈⁺ (M +H+): 1272.5711, found: 1272.5714.

Compound 30: at 0° C., the compound 29 (11 mg, 9.20 μmol) was placed in a solution of THF (1.0 mL), and 1 M LiOH aqueous solution (20 μL) was added. After TLC showed that the starting materials were completely converted, the mixture was diluted with DCM and washed with water and brine. After filtration, the filtrate was concentrated under reduced pressure to afford a crude product. The crude product was separated and purified by silica gel column chromatography (DCM/MeOH v/v 10:1) to afford a semi-deprotected product. The semi-protected product was placed in a solution of THE/MeOH/H₂O/AcOH (v/v/v/v 10:5:4:1, 2 mL), and 10% Pd/C (50 mg) was added. The reaction was stirred under an H₂ atmosphere for 24 h. Subsequently, filtration and concentration under vacuum were carried out, and elution was carried out by Sephadex LH-20 column (H₂O). A two-step reaction was carried out to afford white solid 30 (4.5 mg, 5.88 μmol, 64%). ¹H NMR (600 MHZ, Deuterium Oxide) δ4.98 (s, 1H, 1′-H), 4.84 (s, 1H, 1-H), 4.77 (s, 1H, 1″-H), 4.51 (d, J=3.3 Hz, 1H, 2″-H), 4.41-4.38 (m, 1H, 2′-H), 4.24 (dd, J=11.7, 3.4 Hz, 1H, 2-H), 4.16-4.12 (m, 1H, 3′-H), 4.05 (q, J=6.4 Hz, 1H, 5-H), 4.03-3.98 (m, 3H, 3-H, 4-H, 5″-H), 3.97-3.92 (m, 2H, 5′-H, 3″-H), 3.85-3.81 (m, 2H, 4′-H, 4″-H), 3.78 (dt, J=11.3, 6.4 Hz, 1H, CH₂-Linker), 3.54 (dt, J=11.3, 6.1 Hz, 1H, CH₂-Linker), 3.11 (t, J=7.5 Hz, 2H, CH₂-Linker), 2.20 (s, 3H, CH₃C (N)), 2.05 (s, 3H, CH₃CO), 2.05 (s, 3H, CH₃CO), 2.02-1.97 (m, 2H, CH₂-Linker), 1.99 (s, 3H, CH₃CO), 1.97 (s, 3H, CH₃CO), 1.23 (d, J=6.5 Hz, 3H, 6-H); ¹³C NMR (151 MHz, Deuterium Oxide) δ174.82, 174.26, 174.20, 173.93, 166.06, 163.13, 162.89, 99.90 (C-1′), 98.56 (C-1″), 97.23 (C-1), 78.23, 77.39, 76.93, 75.13, 70.00, 66.54, 65.70, 64.94, 56.20, 51.95, 51.35, 49.49, 47.75, 37.14, 26.74, 21.92, 21.91, 21.80, 21.75, 18.53, 15.35; HR-ESI-MS (m/z): calcd for C₃₁H₅₂N₇O₁₆⁺ (M +H+): 778.3465, found: 778.3466.

### Comparative Example 1

### Synthesis Attempt of Trisaccharide 35

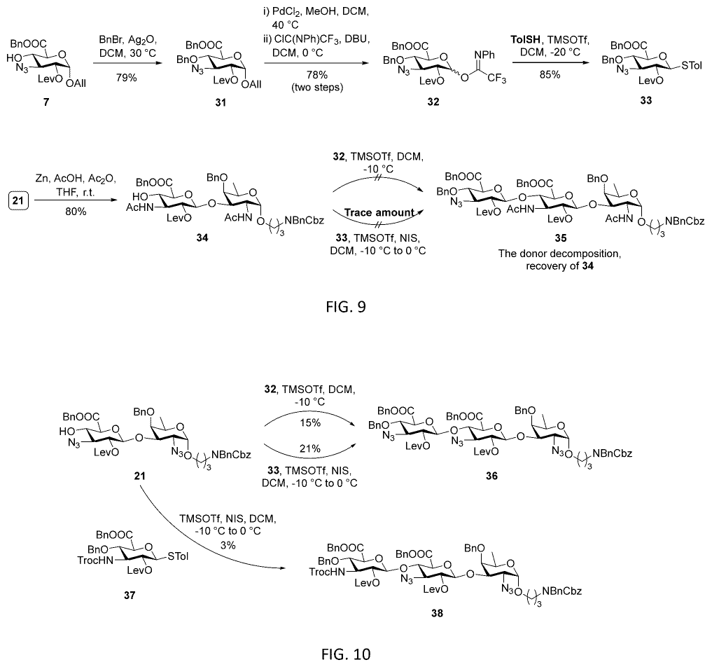

As shown in FIG. 9, taking the intermediate compound 7 as a raw material, the hydroxyl at position C₄ of the compound 7 was protected with a benzyl group under the action of benzyl bromide and silver oxide to afford compound 31. Subsequently, the allyl at the reducing end of the compound 31 was removed with palladium chloride (PdCl₂) to afford a corresponding hemiacetal derivative, which was then converted into N-phenyltrifluoroimine ester donor 32. Under the catalysis of TMSOTf, the compound 32 reacted with p-toluenethiol to afford thioglycoside donor 33. Under the action of zinc powder and acetic anhydride, the intermediate disaccharide 21 reduced the azido at positions C₂ and C_{3′} to aminoacetyl to afford compound 34. Subsequently, taking TMSOTf as an activator, the disaccharide acceptor 34 underwent the glycosylation reaction with the trifluoroacetimidate donor 32 and the thioglycoside donor 33, in an attempt to assemble and synthesize trisaccharide 35.

### Specific Experimental Procedures and Steps

Compound 31: under nitrogen protection, benzyl glucuronide 7 (1.2 g, 2.68 mmol) was dissolved in anhydrous DCM (27 mL), and BnBr (1.59 mL, 13.4 mmol) and Ag20 (1.86 g, 8.04 mmol) were added at 0° C. The reaction mixture was heated to 30° C. and stirred for 24 h. After filtration through Celite and concentration, residues were purified by silica gel column chromatography (petroleum ether/ethyl acetate 10:1 v/v) to afford white solid product 31 (1.14 g, 2.12 mmol, 79%). [α]²⁵_{D}=+68.4° (c 1.00, CH₃Cl); ¹H NMR (400 MHZ, Chloroform-d) δ7.40-7.16 (m, 10H, Ar-H), 5.94-5.81 (m, 1H, All-CH), 5.28 (dq, J=17.2, 1.6 Hz, 1H, All-CH₂), 5.20 (s, 2H, ArCH), 5.07 (d, J=3.6 Hz, 1H, 1-H), 4.75-4.67 (m, 2H, 2-H, ArCH), 4.47 (d, J=10.5 Hz, 1H, ArCH), 4.30 (d, J=9.8 Hz, 1H, 5-H), 4.20 (ddt, J=13.0, 5.3, 1.5 Hz, 1H, All-CH₂), 4.07-4.01 (m, 1H, All-CH₂), 4.00 (t, J=10.1 Hz, 1H, 3-H), 3.66 (t, J=9.7 Hz, 1H, 4-H), 2.88-2.57 (m, 4H, CH₂-Lev), 2.19 (s, 3H, CH₃CO); ¹³C NMR (101 MHZ, Chloroform-d) δ206.06, 171.85, 168.82, 140.90, 137.13, 134.96, 133.01, 128.66, 128.59, 128.51, 128.44, 128.42, 128.17, 128.05, 127.68, 127.00, 118.48, 77.99, 75.07, 71.51, 70.33, 69.14, 67.51, 65.40, 63.04, 37.80, 29.82, 27.86; IR νₘₐₓ (film)=2926, 2108, 1747, 1716, 1455, 1362, 1181, 1154, 1053, 739, 697 cm⁻¹; HR-ESI-MS (m/z): calcd for C₂₈H₃₁N₃O₈Na⁺ (M+Na⁺): 560.2003, found: 560.2005.

Compound 32: compound 31 (0.64 g, 1.19 mmol) was dissolved in MeOH/DCM (2/1, v/v, 24 mL), PdCl₂ (210 mg, 1.19 mmol) was added and the reaction was stirred at 35° C. for 4.5 h. The mixture was filtered and concentrated under vacuum, and separated and purified by silica gel column chromatography (petroleum ether/ethyl acetate 2:1 v/v) to afford corresponding hemiacetal. At 0° C., 2,2,2-trifluoro-N-phenyliminoacetyl chloride (0.89 mL, 5.95 mmol) and DBU (0.53 mL, 3.57 mmol) were added to a solution of DCM (12 mL) of the above-mentioned hemiacetal. The reaction was heated to room temperature and stirred for 3 h. The solution was concentrated under vacuum until residues remained, and the residues were purified by silica gel column chromatography (petroleum ether/ethyl acetate 20:1→10:1 v/v) to afford light yellow syrup 32 (0.62 g, 0.93 mmol, 78%, two-step reaction). [α]²⁵_{D}=+31.1° (c 1.00, CH₃Cl); ¹H NMR (600 MHZ, Chloroform-d) δ7.43-6.76 (m, 15H, Ar-H), 5.23 (d, J=12.2 Hz, 1H,

ArCH), 5.19 (d, J=12.2 Hz, 1H, ArCH), 5.13-5.04 (m, 1H, 2-H), 4.68 (d, J=10.5 Hz, 1H, ArCH), 4.49 (d, J=10.5 Hz, 1H, ArCH), 4.17-3.89 (m, 1H, 5-H), 3.83 (t, J=9.5 Hz, 1H, 4-H), 3.75-3.63 (m, 1H, 3-H), 2.84-2.78 (m, 2H, CH₂-Lev), 2.67-2.62 (m, 2H, CH₂-Lev), 2.18 (s, 3H, CH₃CO); 13C NMR (151 MHz, Chloroform-d) δ205.76, 171.05, 167.20, 142.92, 136.71, 134.69, 128.78, 128.73, 128.70, 128.64, 128.48, 128.25, 128.22, 124.57, 119.25, 94.49, 77.29, 75.54, 75.14, 70.60, 67.81, 65.67, 37.88, 29.67, 27.74; IR νₘₐₓ (film)=2919, 2109, 1749, 1719, 1597, 1489, 1455, 1405, 1365, 1327, 1212, 1161, 1087, 1028, 910, 778, 755, 696 cm⁻¹.

Compound 33: at −20° C., the N-phenyl trifluoroacetimidate donor 32 (0.45 g, 0.6 7mmol) was placed in anhydrous DCM (7 mL), and ToISH (83 mg, 0.67 mmol) and a 4 Å molecular sieve (flame dried) were added. Under an argon atmosphere, TMSOTf (23 μL, 0.134 mmol) was added dropwise. When TLC showed that the starting materials were completely converted, the reactants were quenched with Et₃N, then filtered and concentrated under vacuum. The crude product was separated and purified by silica gel column chromatography (petroleum ether/ethyl acetate v/v 5:1) to afford white solid 33 (343 mg, 0.57 mmol, 85%). [α]²⁵_{D}=−14.32 (c 1.0, CH₃Cl); ¹H NMR (600 MHZ, Chloroform-d) δ7.36 (d, J=6.5 Hz, 5H, Ar-H), 7.30-7.24 (m, 5H, Ar-H), 7.18 (d, J=7.8 Hz, 2H, Ar-H), 7.05 (d, J=7.7 Hz, 2H, Ar-H), 5.23-5.16 (m, 2H, ArCH), 4.81 (dd, J=10.8, 8.9 Hz, 1H, 2-H), 4.67 (d, J=10.5 Hz, 1H, ArCH), 4.56 (d, J=9.9 Hz, 1H, 1-H), 4.49 (d, J=10.5 Hz, 1H, ArCH), 3.95 (d, J=9.5 Hz, 1H, 5-H), 3.70 (t, J=9.6 Hz, 1H, 4-H), 3.62 (t, J=9.7 Hz, 1H, 3-H), 2.83 (td, J=6.6, 2.1 Hz, 2H, Lev-H), 2.69 (q, J=6.9 Hz, 2H, Lev-H), 2.32 (s, 3H,

Lev-CH₃), 2.21 (d, J=1.8 Hz, 3H, Tol (CH₃)); ¹³C NMR (151 MHZ, Chloroform-d) δ205.94, 171.15, 167.25, 138.74, 136.97, 134.92, 133.76, 129.72, 128.66, 128.62, 128.53, 128.43, 128.21, 128.16, 128.10, 127.64, 87.00, 78.55, 77.55, 75.06, 70.04, 67.87, 67.56, 37.83, 29.84, 27.99, 21.18; IR νₘₐₓ (film)=2919, 2107, 1748, 1718, 1494, 1455, 1401, 1363, 1262, 1201, 1174, 1150, 1072, 1020, 910, 810, 752, 698, 580 cm⁻¹; HR-ESI-MS (m/z): calcd for C₃₂H₃₃N₃O₇SNa⁺ (M+Na⁺): 626.1932, found: 626.1929.

Compound 34: at room temperature, the compound 21 (1.70 g, 1.60 mmol) was placed in a solution of THF (16 mL) and stirred, then AcOH (91 μL, 1.60 mmol) and TBAF (2.4 mL, 2.4 mmol) were added, and stirring was carried out for 5 h. An organic phase was dried over anhydrous Na₂SO₄ and concentrated under vacuum. The crude product was separated and purified by silica gel column chromatography (PE/EA v/v 4:1→3:2) to afford colorless syrup 34 (1.40 g, 1.47 mmol, 92%). [α]²⁵_{D}=+37.8° (c 1.00, CH₃Cl); ¹H NMR (600 MHZ, Chloroform-d) δ7.40-7.14 (m, 20H, Ar-H), 6.71-6.57 (m, 2H, NH), 5.31-5.14 (m, 4H, ArCH), 4.88 (d, J=11.6 Hz, 1H, ArCH), 4.78 (t, J=7.1 Hz, 1H, 2′-H), 4.75 (d, J=6.4 Hz, 1H, 1′-H), 4.67-4.56 (m, 3H, 2-H, 1-H, ArCH), 4.53 (d, J=11.6 Hz, 1H, ArCH), 4.40 (d, J=15.9 Hz, 1H, ArCH), 4.07 (d, J=9.2 Hz, 1H, 5′-H), 4.01-3.90 (m, 2H, 3′-H, 4′-H), 3.83-3.71 (m, 2H, 5-H, 3-H), 3.69-3.57 (m, 3H, 4-H, CH₂-Linker), 3.39-3.22 (m, 2H, CH₂-Linker), 2.90 (ddd, J=18.4, 9.8, 4.4 Hz, 1H, CH₂-Lev), 2.81-2.71 (m, 1H, CH₂-Lev), 2.69-2.55 (m, 2H, CH₂-Lev), 2.15 (s, 3H, CH₃CO), 2.04 (s, 3H, CH₃CO), 2.01 (s, 3H, CH₃CO), 1.81-1.58 (m, 2H, CH₂-Linker), 1.08 (d, J=6.4 Hz, 3H, 6-H); ¹³C NMR (151 MHZ, Chloroform-d) δ206.81, 174.23, 173.56, 170.80, 168.55, 156.43, 138.80, 137.42, 136.46, 135.27, 128.69, 128.58, 128.41, 128.28, 128.14, 128.13, 127.88, 127.69, 127.52, 127.25, 101.48 (C-1′), 97.88 (C-1), 79.69, 78.94, 75.78, 74.92, 73.02, 70.37, 67.43, 67.20, 66.74, 63.58, 57.57, 49.72, 48.34, 42.92, 38.04, 29.68, 27.69, 27.25, 23.38, 22.72, 16.58; IR νₘₐₓ (film) 3305, 2924, 1747, 1661, 1549, 1454, 1423, 1372, 1218, 1177, 1127, 1072, 1042, 751, 699, 593 cm⁻¹; HR-ESI-MS (m/z): calcd for C₅₃H₆₃N₃O₁₅Na⁺ (M+Na⁺): 1004.4151, found: 1004.4157.

### Compound 35

Condition I: at −10° C., the donor 32 (21 mg, 0.0306 mmol), the acceptor 34 (20 mg, 0.0204 mmol) and a 4 Å molecular sieve (flame dried) were placed in a solution of dried DCM (1.5 mL) and stirred. Under argon protection, TMSOTf (1.1 μL, 0.006 mmol) was added dropwise. When TLC detection showed that the glycosyl donor was completely converted, Et₃N was added dropwise to quench the reaction, then filtration and concentration under vacuum were carried out. The crude product was separated and purified by silica gel column chromatography and detected by MALDI-TOF and NMR. Unreacted acceptor and donor degradation products indicate that the glycosylation reaction failed.

Condition II: at −20° C., the thioglycoside donor 33 (18 mg, 0.0306 mmol), the acceptor 34 (20 mg, 0.0204 mmol) and a 4 Å molecular sieve were placed in dried DCM (1.5 mL) and stirred, and NIS (8.3 mg, 0.0367 mmol) and TMSOTf (1.1 μL, 0.006 mmol) were added. The reaction was stirred at 0° C. for 5 h until TLC detection showed that the glycosyl donor was completely converted. The reaction solution was diluted with DCM and filtered. The filtrate was washed with 10% (w/v) Na₂S₂O₃, a saturated NaHCO₃ solution, and brine. An organic phase was dried over anhydrous Na₂SO₄ and concentrated under vacuum. The crude product was separated and purified by silica gel column chromatography to afford trace products only. A large number of unreacted acceptor residues indicate that the glycosylation reaction failed.

### Comparative Example 2

### Synthesis Attempt of Trisaccharide 38

As shown in FIG. 10, in order to improve the reactivity of a disaccharide

acceptor, an attempt was made to assemble trisaccharide by using disaccharide 8, which was not reduced by azide, as acceptor. Firstly, an attempt was made to take trifluoroacetimidate 32 and thioglycoside 33 as glycosyl donors and allow them to underwent the glycosylation reaction with a disaccharide acceptor 8 to afford trisaccharides 27 with a β-configuration only, with yields of 15% and 21%, respectively. Although the yield is not ideal, the reactivity of the disaccharide acceptor was improved, and the reactivity of the thioglycoside donor is higher than that of the trifluoroacetimidate donor. In order to realize the orthogonal modification of the amino at position C_{3″,} we attempted to carry out the glycosylation reaction between thioglycoside donor 37 and disaccharide acceptor 21 at −10° C. to 0° C. by taking TMSOTf and NIS as activators, in an attempt to synthesize trisaccharide 38. However, the yield of target trisaccharide 29 was not ideal, which was only 3%. The reaction resulted in byproducts from donor decomposition and the glycosyl acceptor 21.

### Specific Experimental Procedures and Steps

Compound 36: condition I: at −10° C., the donor 32 (21 mg, 0.0316 mmol), the acceptor 21 (20 mg, 0.0211 mmol) and a 4 Å molecular sieve (flame dried) were placed in a solution of dried DCM (1.6 mL) and stirred. Under argon protection, TMSOTf (1.1 μL, 6.32 μmol) was added dropwise. When TLC detection showed that the glycosyl donor was completely converted, Et₃N was added dropwise to quench the reaction, then filtration and concentration under vacuum were carried out. The crude product was separated and purified by silica gel column chromatography (PE/EA v/v 5:1→2:1) to afford colorless syrup 3 6(4.5 mg, 4.74 μmol, 15%).

Condition II: at −20° C., the thioglycoside donor 33 (19 mg, 0.0316 mmol), the acceptor 21 (20 mg, 0.0211 mmol) and a 4 Å molecular sieve were placed in dried DCM (1.5 mL) and stirred, and NIS (8.5 mg, 0.038 mmol) and TMSOTf (1.1 μL, 6.62 μmoL) were added. The reaction was stirred at 0° C. for 5 h until TLC detection showed that the glycosyl donor was completely converted. The reaction solution was diluted with DCM and filtered. The filtrate was washed with 10% (w/v) Na₂S₂O₃, a saturated NaHCO₃ solution, and brine. An organic phase was dried over anhydrous Na₂SO₄ and concentrated under vacuum. The crude product was separated and purified by silica gel column chromatography (PE/EA v/v 5:1→2:1) to afford colorless syrup 36 (9.5 mg, 6.64 μmol, 21%). [α]²⁵_{D}=+18.9° (c 0.50, CH₃Cl); ¹H NMR (600 MHZ, Chloroform-d) δ7.38-7.14 (m, 30H, Ar-H), 5.32 (d, J=11.8 Hz, 1H, ArCH), 5.20-5.13 (m, 4H, ArCH), 5.11 (d, J=11.9 Hz, 1H, ArCH), 4.85 (dd, J=10.4, 7.9 Hz, 1H, 2′-H), 4.80 (d, J=11.4 Hz, 1H, ArCH), 4.82-4.74 (m, 1H, 1-H), 4.75 (dd, J=10.3, 7.9 Hz, 1H, 2″-H), 4.73-4.66 (m, 1H, 1′-H), 4.62 (d, J=10.7 Hz, 1H, ArCH), 4.55-4.46 (m, 2H, ArCH), 4.44 (J=10.4 Hz, 1H, ArCH), 4.44 (d, J=8.3 Hz, 1H, 1″-H), 4.40 (d, J=11.5 Hz, 1H, ArCH), 4.14-4.07 (m, 1H, 5′-H), 3.98 (t, J=9.5 Hz, 1H, 4′-H), 3.96-3.88 (m, 1H, 3-H), 3.77 (d, J=9.6 Hz, 1H, 5″-H), 3.71-3.75 (m, 1H, 5-H), 3.69 (t, J=9.6 Hz, 1H, 4″-H), 3.67-3.64 (m, 2H, 2-H), 3.64-3.58 (m, 1H, CH₂-Linker), 3.59-3.54 (m, 2H, 3′-H, 4-H), 3.48-3.35 (m, 2H, CH₂-Linker), 3.31 (dd, J=11.6, 8.2 Hz, 2H, 3″-H, CH₂-Linker), 2.87-2.50 (m, 8H, CH₂-Lev), 2.19 (s, 3H, CH₃CO), 2.16 (s, 3H, CH₃CO), 1.89-1.73 (m, 2H, CH₂-Linker), 1.04-0.94 (m, 3H, 6-H); ¹³C NMR (151 MHZ, Chloroform-d) δ206.17, 205.93, 171.23, 167.06, 166.88, 138.43, 137.83, 137.04, 134.82, 134.71, 130.04, 129.74, 129.26, 129.19, 128.90, 128.71, 128.65, 128.64, 128.58, 128.40, 128.19, 128.10, 128.04, 127.84, 127.62, 127.42, 127.25, 102.54 (C-1′), 100.31 (C-1″), 98.15 (C-1), 78.85, 77.65, 76.66, 75.21, 74.93, 74.91, 74.69, 70.96, 70.63, 67.72, 67.26, 66.39, 65.83, 65.80, 64.61, 59.38, 50.59, 37.95, 37.70, 29.87, 29.86, 27.77, 27.60, 27.23, 27.17, 24.74, 22.70, 16.38; IR νₘₐₓ (film) 2918, 2109, 1749, 1715, 1372, 1218, 1146, 1042, 751, 699 cm⁻¹; HR-ESI-MS (m/z): calcd for C₇₄H₈₀N₁₀O₂₀Na⁺ (M+Na⁺): 1451.5443, found: 1451.5451.

Compound 37: under nitrogen protection, zinc powder (106 mg, 1.66 mmol) was added to a solution of AcOH/THF (1:2 v/v, 3 mL) in which the compound 33 (50 mg, 0.083 mmol) was dissolved. The mixture was stirred at 37° C. for 12 h. The solution was filtered and concentrated under vacuum to afford a crude product. The crude product was dissolved in THF (7 mL) and pyridine (20 μL, 0.249 mmol, 3 eq), and then a solution of TrocCl (11.4 μL, 0.083 mmol) in THF (1 mL) was added dropwise at 0° C. The reaction mixture was stirred at 0° C. for 2 h until TLC showed that the starting materials were completely converted. Then, MeOH was added at 0° C. for quenching. The mixture was concentrated under vacuum until residues remained, and the residues were purified by silica gel column chromatography (petroleum ether/ethyl acetate 5:1→2:1 v/v) to afford product 37 (19 mg, 0.026 mmol, 31%) as colorless syrup. [α]²⁵_{D}=−25.3° (c 1.00, CH₃Cl); ¹H NMR (600 MHZ, Chloroform-d) δ7.47-6.94 (m, 14H, Ar-H), 5.21 (s, 2H, CH₂-Troc), 4.88 (d, J=9.4 Hz, 1H, NH), 4.76 (d, J=12.1 Hz, 1H, CH₂-Troc), 4.75 (t, J=9.7 Hz, 1H, 2-H), 4.70 (d, J=12.1 Hz, 1H, CH₂-Troc), 4.64 (d, J=9.8 Hz, 1H, 1-H), 4.48 (d, J=11.0 Hz, 1H, ArCH), 4.44 (d, J=11.1 Hz, 1H, ArCH), 4.03 (d, J=9.4 Hz, 1H, 5-H), 3.94 (q, J=9.8

Hz, 1H, 3-H), 3.74 (t, J=9.7 Hz, 1H, 4-H), 2.81-2.68 (m, 2H, CH₂-Lev), 2.64-2.57 (m, 2H, CH₂-Lev), 2.33 (s, 3H, CH₃CO), 2.18 (s, 3H, ArCH₃); ¹³C NMR (151 MHZ, Chloroform-d) δ205.86, 171.78, 167.67, 154.25, 138.82, 137.05, 134.95, 134.03, 129.71, 128.66, 128.60, 128.53, 128.41, 128.11, 128.03, 127.22, 95.47, 86.74, 79.10, 76.52, 74.64, 74.26, 69.82, 67.53, 58.36, 37.80, 29.78, 27.97, 21.21; IR νₘₐₓ (film) 3334, 2923, 1742, 1719, 1544, 1495, 1455, 1401, 1364, 1317, 1288, 1257, 1205, 1157, 1074, 1029, 814, 735, 698 cm⁻¹; HR-ESI-MS (m/z): calcd for C₃₅H₃₆Cl₃NO₉SNa⁺ (M+Na⁺): 774.1069, found: 774.1072.

Compound 38: at −10° C., the thioglycoside donor 37 (24 mg, 0.0316 mmol), the acceptor 21 (20 mg, 0.0211 mmol) and a 4 Å molecular sieve were placed in dried DCM (1.6 mL) and stirred, and NIS (9 mg, 0.038 mmol) and TMSOTf (1.1 μL, 6.32 μmoL) were added. The reaction was stirred at 0° C. for 5 h until TLC detection showed that the glycosyl donor was completely converted. The reaction solution was diluted with DCM and filtered. The filtrate was washed with 10% (w/v) Na₂S₂O₃, a saturated NaHCO₃ solution, and brine. An organic phase was dried over anhydrous Na₂SO₄ and concentrated under vacuum. The crude product was separated and purified by silica gel column chromatography (PE/EA v/v 3:1→1:1) to afford colorless syrup 38 (1.5 mg, 0.95 μmol, 3%). ¹H NMR (600 MHZ, Chloroform-d) δ7.38-7.10 (m, 30H, Ar-H), 5.35 (d, J=11.8 Hz, 1H, ArCH), 5.21-5.12 (m, 4H, ArCH), 5.07 (d, J=11.8 Hz, 1H, ArCH), 4.86 (dd, J=10.3, 7.9 Hz, 1H, 2′-H), 4.83-4.74 (m, 3H, 1-H, CH₂-Troc), 4.76-4.70 (m, 2H, 2″-H, ArCH), 4.70-4.65 (m, 1H, 1′-H), 4.55-4.48 (m, 2H, ArCH), 4.46-4.39 (m, 4H, 1″-H, ArCH), 4.06-3.97 (m, 2H), 3.97-3.87 (m, 1H), 3.81 (q, J=9.8 Hz, 1H), 3.79-3.70 (m, 3H), 3.66 (dd, J=10.9, 3.7 Hz, 1H), 3.66-3.61 (m, 1H, CH₂-Linker), 3.59-3.54 (m, 2H), 3.48-3.29 (m, 3H, CH₂-Linker), 2.85-2.65 (m, 6H, CH₂-Lev), 2.58-2.46 (m, 2H, CH₂-Lev), 2.17 (s, 3H, CH₃CO), 2.16 (s, 3H, CH₃CO), 1.90-1.71 (m, 2H, CH₂-Lev), 1.03-0.95 (m, 3H, 6-H); HR-ESI-MS (m/z): calcd for C₇₇H₈₃Cl₃N₈O₂₂Na⁺ (M+Na⁺): 1599.4580, found: 1599.4586.

### Comparative Example 3

### Synthesis Attempt of Trisaccharide 43 and 44

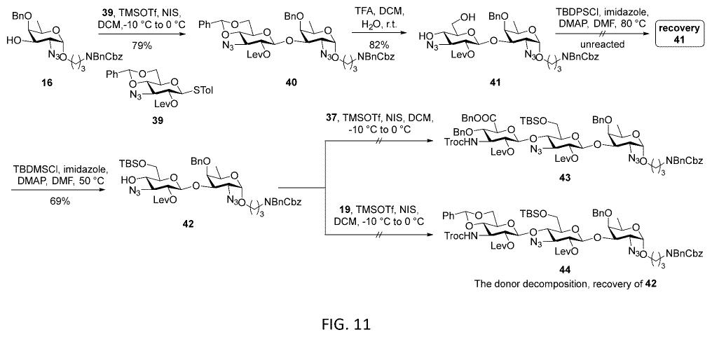

As shown in FIG. 11, under the action of TMSOTf and N-iodosuccinimide (NIS), acceptor 16 and thioglycoside donor 39 underwent the glycosidation reaction at −10° C. to 0° C. to afford disaccharide 39 with complete β selectivity, with a yield of 79%. 4,6-O-benzylidene of compound 40 was hydrolyzed with trifluoroacetic acid (TFA) to afford diol 41 with a yield of 82%. Subsequently, when selective protection was performed on the hydroxyl at position C_{6′} with tert-butyldimethylsilyl (TBDPS), a desired product could not be afforded even though the reaction was carried out at 80° C. Considering that the hydroxyl at position C_{6′} can be selectively protected with tert-butyldimethylsilyl (TBS), the influence of spatial steric hindrance is reduced. Subsequently, the reaction was carried out at 50° C. to successfully afford product 42 with a yield of 69%. Unfortunately, even if the equivalent of a catalyst TMSOTf was increased to 0.6, the glycosylation reaction between glycosyl donor 37 and disaccharide acceptor 42 still failed. In addition, the glycosidation reaction between donor 19 with higher reactivity and disaccharide acceptor 42 cannot result in desired trisaccharides. Through the analysis of decomposed donor by-products and unreacted acceptors, the failure of the above-mentioned glycosylation reaction may be caused by the steric hindrance of TBS at position O6.

### Specific Experimental Procedures and Steps

Compound 40: under argon protection and at −10° C., NIS (330 mg, 1.46 mmol) and TMSOTf (44 μL, 0.244 mmol) were added to a mixed solution of anhydrous DCM (6.8 mL) in which sulfur donor 39 (610 mg, 1.22 mmol), acceptor 16 (380 mg, 0.68 mmol), and newly activated 4 Å MS were dissolved. The reaction mixture was stirred at 0° C. for 4.5 h until TLC showed complete conversion. The mixture was diluted with DCM and filtered, and the filtrate was washed with a 10% (w/v) Na₂S₂₀₃ solution, saturated NaHCO₃ and brine in turn. An organic layer was dried over anhydrous Na₂SO_{4,} filtered, and concentrated under vacuum. The residues were purified by silica gel column chromatography (petroleum ether/ethyl acetate 5:1→2:1 v/v) to afford product 40 (501 mg, 0.54 mmol, 79%). [α]²⁵_{D}=+28.0° (c 1.00, CH₃Cl); ¹H NMR (400 MHZ, Chloroform-d) δ7.56-7.09 (m, 20H, Ar-H), 5.59 (s, 1H, ArCH), 5.23-5.12 (m, 1H, ArCH), 4.96 (dd, J=9.9, 7.8 Hz, 1H, 2′-H), 4.88 (d, J=11.5 Hz, 1H, ArCH), 4.86-4.80 (m, 1H, 1-H), 4.80-4.76 (m, 1H, 1′-H), 4.62-4.31 (m, 4H, ArCH, 6′-H), 4.06-3.93 (m, 1H, CH₂-Linker), 3.88-3.74 (m, 3H, 3′-H, 6′-H, 5-H), 3.57-3.26 (m, 5H, 4′-H, 5′-H, CH₂-Linker, 3-H, 4-H), 3.56-3.41 (m, 2H, 2-H, CH₂-Linker), 3.37-3.30 (m, 1H, CH₂-Linker), 2.91-2.63 (m, 4H, CH₂-Lev), 2.16 (s, 3H, CH₃CO), 1.90-1.82 (m, 2H, CH₂-Linker), 1.14-1.05 (m, 3H, 6-H); ¹³C NMR (101 MHZ, Chloroform-d) δ206.01, 171.40, 138.40, 137.82, 136.50, 129.28, 128.69, 128.60, 128.51, 128.39, 128.35, 128.22, 128.03, 127.83, 127.72, 127.42, 127.23, 126.02, 102.63 (C-1′), 101.60, 98.34 (C-1), 79.41, 77.35, 77.35, 77.03, 76.72, 74.95, 71.90, 68.52, 67.27, 66.52, 65.89, 63.11, 59.54, 50.49, 37.92, 29.77, 27.75, 16.44; IR νₘₐₓ (film) 2933, 2108, 1753, 1698, 1497, 1454, 1421, 1360, 1315, 1218, 1125, 1092, 1043, 1002 cm⁻¹; HR-ESI-MS (m/z): calcd for C₄₉H₅₅N₇O₁₂Na⁺ (M+Na⁺): 956.3801, found: 956.3805.

Compound 41: trifluoroacetic acid (0.5 mL, 6.72 mmol) and H₂O (88 μL, 4.90 mmol, 10 eq.) were added to a solution of DCM (5.0 mL) in which the compound 40 (460 mg, 0.49 mmol) was dissolved. The reaction mixture was stirred at room temperature for 4 h until TLC showed that the starting materials were completely converted. The mixture was diluted with DCM, and washed with water, a saturated NaHCO₃ solution, and saturated brine. The mixture was dried over Na₂SO₄ and concentrated under vacuum. The residues were purified by silica gel column chromatography (DCM/MeOH 50:1→20:1 v/v) to afford compound 41 (340 mg, 0.402 mmol, 82%) as colorless syrup. [α]²⁵_{D}=+27.2° (c 1.00, CH₃Cl); ¹H NMR (600 MHZ, Chloroform-d) δ7.46-7.13 (m, 15H, Ar-H), 5.22-5.10 (m, 2H, ArCH), 4.91 (dd, J=9.7, 7.8 Hz, 1H, 2′-H), 4.88 (d, J=11.6 Hz, 1H, ArCH), 4.86-4.77 (m, 1H, 1-H), 4.77-4.69 (m, 1H, 1′-H), 4.59 (d, J=11.6 Hz, 1H, ArCH), 4.57-4.41 (m, 2H, ArCH), 4.12-3.94 (m, 1H, 3-H), 3.90-3.74 (m, 3H, 5-H, 6′-H), 3.72 (dd, J=10.8, 3.6 Hz, 1H, 2-H), 3.67-3.53 (m, 4H, 4-H, CH₂-Linker, 3′-H, 4′-H), 3.49-3.38 (m, 2H, 5′-H, CH₂-Linker), 3.38-3.27 (m, 1H, CH₂-Linker), 2.84-2.66 (m, 4H, CH₂-Lev), 2.15 (s, 3H, CH₃CO), 1.90-1.74 (m, 2H, CH₂-Linker), 1.10 (d, J=6.4 Hz, 3H, 6-H); ¹³C NMR (151 MHz, Chloroform-d) δ206.31, 171.52, 156.38, 138.44, 137.68, 136.49, 128.60, 128.56, 128.50, 128.20, 128.05, 127.80, 127.69, 127.42, 127.20, 101.86 (C-1), 98.27 (C-1′), 78.86, 77.46, 76.10, 74.80, 71.83, 69.75, 67.35, 66.83, 66.60, 65.38, 62.29, 59.77, 50.31, 44.34, 37.87, 29.74, 28.22, 16.47; IR νₘₐₓ (film) 3462, 2935, 2107, 1750, 1696, 1497, 1454, 1423, 1362, 1314, 1220, 1161, 1126, 1044 cm⁻¹; HR-ESI-MS (m/z): calcd for C₄₂H₅₁N₇O₁₂Na⁺ (M+Na⁺): 868.3488, found: 868.3493.

Compound 42: tert-butyldimethylsilyl chloride (30 mg, 0.20 mmol), imidazole (23 mg, 0.33 mmol), and 4-dimethylaminopyridine (3 mg, 0.02 mmol) were added to a solution of anhydrous DMF (0.3 mL) in which 41 (140 mg, 0.166 mmol) was dissolved. The reaction mixture was stirred overnight at 50° C., and then the reaction was quenched by adding methanol (3 mL). The mixture was diluted with ethyl acetate (30 mL) and washed with water, a saturated NaHCO₃ solution, and saturated brine. A merged organic phase was dried over Na₂SO₄, filtered, and concentrated. The crude product was purified by rapid silica column chromatography (petroleum ether/ethyl acetate 10:1 v/v) to afford 42 (110 mg, 0.115 mmol, 69%) as colorless oil. [α]²⁵_{D}=+19.7° (c 1.00, CH₃Cl); ¹H NMR (600 MHZ, Chloroform-d) δ7.40-7.15 (m, 15H, Ar-H), 5.22-5.14 (m, 2H, ArCH), 4.92-4.88 (m, 2H, ArCH, 2′-H), 4.84-4.76 (m, 1H, 1-H), 4.73-4.65 (m, 1H, 1′-H), 4.57-4.43 (m, 3H, ArCH), 4.02-3.93 (m, 1H, 3-H), 3.93-3.86 (m, 1H, 6′-H), 3.86-3.73 (m, 1H, 5-H), 3.81 (dd, J=10.3, 6.0 Hz, 1H, 6′-H), 3.69 (dd, J=10.8, 3.6 Hz, 1H, 2-H), 3.68-3.65 (m, 1H, 4-H), 3.66-3.54 (m, 3H, CH₂-Linker, 3′-H, 4′-H), 3.51-3.30 (m, 4H, 5′-H, CH₂-Linker), 2.86-2.68 (m, 4H, CH₂-Lev), 2.16 (s, 3H, CH₃CO), 1.91-1.74 (m, 2H, CH₂-Linker), 1.10 (d, J=6.6 Hz, 3H, 6-H), 0.89 (s, 9H, SiC (CH₃) 3), 0.12-0.06 (m, 6H, Si (CH₃) 2); ¹³C NMR (151 MHZ, Chloroform-d) δ206.09, 171.50, 156.23, 138.53, 137.81, 136.60, 128.58, 128.50, 128.15, 128.02, 127.82, 127.59, 127.38, 127.21, 102.01 (C-1′), 98.33 (C-1), 79.05, 77.84, 75.03, 74.71, 72.15, 71.59, 67.27, 66.60, 66.52, 65.82, 64.41, 59.61, 50.55, 44.67, 37.92, 29.75, 27.80, 25.78, 18.17, 16.42, −5.49; IR νₘₐₓ (film) 3419, 2929, 2108, 1754, 1704, 1472, 1455, 1423, 1361, 1317, 1253, 1153, 1130, 1047, 970 cm⁻¹; HR-ESI-MS (m/z): calcd for C₄₈H₆₅N₇O₁₂SiNa⁺ (M+Na⁺): 982.4353, found: 982.4355.

Compound 43: at −10° C. and under argon protection, NIS (11 mg, 0.05 mmol) and TMSOTf (1.5 μL, 8.4 μmoL) were added to a mixture of anhydrous DCM (0.5 mL) in which donor 37 (32 mg, 0.042 mmol), disaccharide acceptor 42 (20 mg, 0.021 mmol), and newly activated 4 Å MS were dissolved. The reaction mixture was stirred at 0° C. for 10 h until TLC showed that the glycosyl donor was completely converted. The reaction was quenched with Et₃N, then filtered and concentrated under vacuum. The residues were purified by silica gel column chromatography and analyzed by MALDI-TOF and NMR. Unreacted acceptor and donor degradation products indicate that the glycosylation reaction failed.

Compound 44: at −10° C. and under argon protection, NIS (11 mg, 0.05 mmol) and TMSOTf (1.5 μL, 8.4 μmoL) were added to a mixture of anhydrous DCM (0.5 mL) in which donor 19 (21 mg, 0.042 mmol), disaccharide acceptor 42 (20 mg, 0.021 mmol), and newly activated 4 Å MS were dissolved. The reaction mixture was stirred at 0° C. for 8 h until TLC showed that the glycosyl donor was completely converted. The reaction was quenched with Et₃N, then filtered and concentrated under vacuum. The residues were purified by silica gel column chromatography and analyzed by MALDI-TOF and NMR. Unreacted acceptor and donor degradation products indicate that the glycosylation reaction failed.

The disclosure has been described by way of example in combination with specific embodiments above. Obviously, the implementation of the disclosure is not limited by the above-mentioned way, as long as various improvements made by adopting the method conception and technical solution of the disclosure, or direct application of the conception and technical solution of the disclosure to other occasions without improvements are all within the protection scope of the disclosure.

What is claimed is:

1. A chemical synthesis method for an oligosaccharide fragment of Pseudomonas aeruginosa serotype O5 O-antigen trisaccharide assembled with a linker arm, wherein the structure of the oligosaccharide fragment of the P. aeruginosa serotype O5 O-antigen trisaccharide assembled with the linker arm is represented by general formula VI: 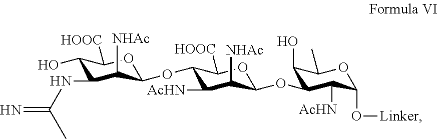
wherein Linker is -L-NH₂, L representing a linker arm; the linker arm L is a chain structure with 2 to 40 carbon atoms that contain 0 to 6 heteroatoms, a substituted or unsubstituted three-six membered cyclic structure, an amide bond, or ureido;
wherein three monosaccharide building blocks A, B, and C are taken as raw materials, as represented by formulas (I) to (III), respectively: 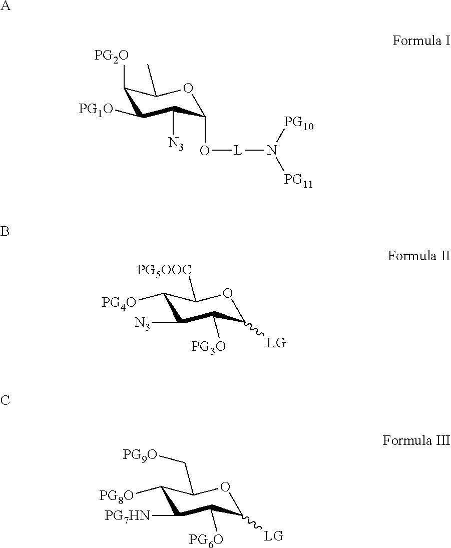
wherein:
PG₁ is H;
PG₂, PG₃, PG₄, and PG₆ are temporary hydroxyl protecting groups, each independently selected from acetyl, benzoyl, pivaloyl, chloracetyl, levulinyl, 9-fluorenylmethoxycarbonyl, allyloxycarbonyl, 2-naphthylmethyl, p-methoxybenzyl, allyl, tert-butyldimethylsilyl, or triethylsilyl;
PG₅ is a carboxyl protecting group, selected from benzyl, methyl, ethyl, tert-butyl, and allyl;
PG₇ is a temporary amino protecting group, selected from trichloroethoxycarbonyl, phthaloyl, 9-fluorenylmethoxycarbonyl, and tert-butyloxycarbonyl;
PG₈ and PG₉ are hydroxyl protecting groups, selected from phenylmethylene, naphthylmethylene, and isopropylidene ketal;
PG₁₀ and PG₁₁ are amino protecting groups, selected from benzyl and carbobenzoxy; and
LG is a leaving group used for a glycosylation reaction, selected from fluorine, chlorine, bromine, iodine, trichloroacetimidate, N-phenyl trifluoroacetimidate glycoside, ethylthio, phenylthio, p-tolylthio, ethylthio, or dibutylphosphonato;
wherein the chemical synthesis method comprises:
(1) carrying out a glycosylation reaction on the monosaccharide building block B and the monosaccharide building block A, and carrying out synthesis to obtain a disaccharide fragment represented by formula IV, 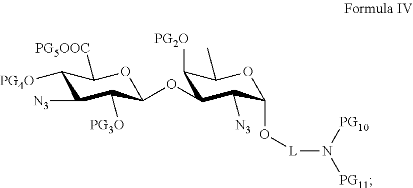
(2) deprotecting the resulting disaccharide fragment, removing the protecting group PG₄, then carrying out glycosylation with the monosaccharide building block C, and carrying out synthesis to obtain a trisaccharide intermediate fragment represented by formula V, 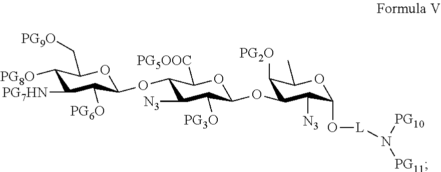
and (3) reducing the resulting trisaccharide intermediate fragment, deprotecting PG₃ and PG₆, and carrying out azidation on deprotected position 2 of the monosaccharide building block B and deprotected position 2 of the monosaccharide building block C; subsequently, reducing positions 4 and 6 of the monosaccharide building block C in the trisaccharide intermediate fragment, removing PG₈ and PG₉, and carrying out oxidization on position 6 to obtain carboxylic acid groups; then, reducing the deprotected position 2 in the monosaccharide building block B and an azide group at position 2 in the monosaccharide building block C to obtain acetylamino; and then, constructing an acetamidine structure with an imine reagent at position 3 in the monosaccharide building block C, and finally, reducing amino in Linker to obtain a target product represented by formula VI.

2. The chemical synthesis method according to claim 1, wherein a synthetic route is as follows:

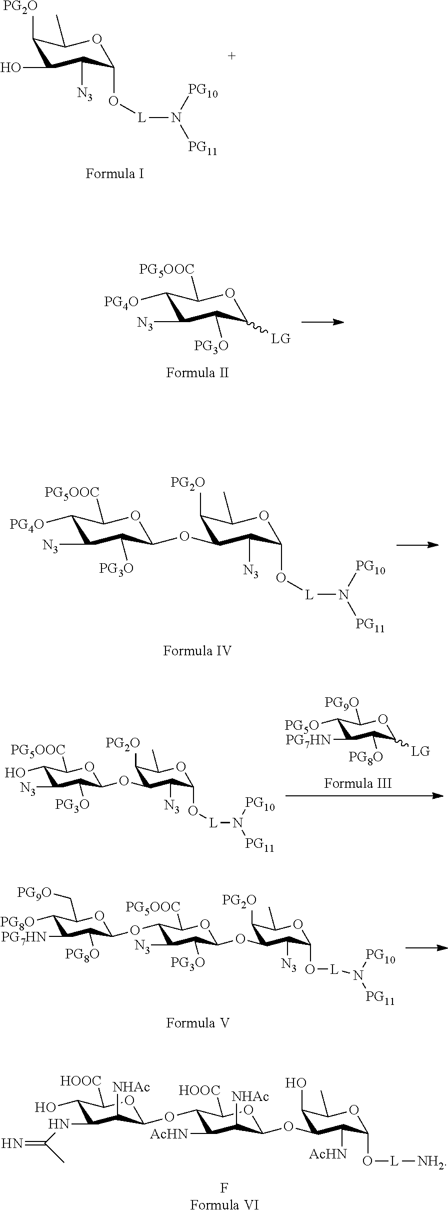

3. The chemical synthesis method according to claim 1, wherein the glycosylation reaction in step (1) is carried out under an atmosphere where an activating reagent Lewis acid, a solvent, and a molecular sieve are present.

4. The chemical synthesis method according to claim 3, wherein the activating reagent Lewis acid for the glycosylation reaction in step (1) is selected from any one or more of borontrifluoride diethyl etherate, trifluoromethanesulfonic acid, trimethylsilyl trifluoromethanesulfonate, and silver trifluoromethanesulfonate.

5. The chemical synthesis method according to claim 1, wherein the glycosylation reaction in step (2) is carried out under an atmosphere where an activating reagent Lewis acid, NIS, a solvent, and a molecular sieve are present.

6. The chemical synthesis method according to claim 5, wherein a dosage of the activating reagent Lewis acid for the glycosylation reaction in step (2) is 0.1 equivalent to 1 equivalent, and a dosage of NIS is 1.2 equivalent to 1.5 equivalent.

7. The chemical synthesis method according to claim 5, wherein the activating reagent Lewis acid for the glycosylation reaction in step (2) is selected from any one or more of borontrifluoride diethyl etherate, trifluoromethanesulfonic acid, trimethylsilyl trifluoromethanesulfonate, and silver trifluoromethanesulfonate.

8. A compound of P. aeruginosa serotype O5 O-antigen trisaccharide assembled with an amino linker arm, wherein the compound is prepared by the method according to claim 1, and the structure is as follows: [chemical structure]
